(19) United States
(12) Patent Application Publication
(10) Pub. No.: US 2024/0122945 A1
(43) Pub. Date: Apr. 18, 2024

(54) USE OF NEUROACTIVE STEROIDS FOR TREATMENT OF SEXUAL DYSFUNCTION

(71) Applicant: Sage Therapeutics, Inc., Cambridge, MA (US)

(72) Inventors: Vijayveer BONTHAPALLY, Cambridge, MA (US); Ellison SUTHOFF, Cambridge, MA (US); Handan GUNDUZ-BRUCE, Lexington, MA (US); Ryan ARNOLD, Cambridge, MA (US)

(21) Appl. No.: 18/263,189

(22) PCT Filed: Jan. 27, 2022
(86) PCT No.: PCT/US2022/014075

§ 371 Date: Jul. 27, 2023

Related U.S. Application Data
(60) Provisional application No. 63/142,833, filed on Jan. 28, 2021.
(60) Provisional application No. 63/142,840, filed on Jan. 28, 2021.

(51) Int. Cl. A61K 31/58; A61P 15/00

(52) U.S. Cl. CPC A61K 31/58; A61P 15/00

(57) ABSTRACT

This invention relates to methods of treating sexual dysfunction and/or maintaining sexual function in a patient by administering to the patient neuroactive steroids.

### CROSS-REFERENCE TO RELATED APPLICATIONS

This application claims the benefit of U.S. Provisional Application No. 63/142,840, filed on Jan. 28, 2021, and U.S. Provisional Application No. 63/142,833, filed on Jan. 28, 2021. The entire contents of the aforementioned applications are incorporated herein by reference in their entireties.

### FIELD OF THE INVENTION

The present invention relates to methods of treating sexual dysfunction, such as treatment-induced sexual dysfunction, and/or maintaining sexual function in patients by administering Compound (1) or Compound (2) as described herein.

### BACKGROUND

Sexual dysfunction (SD) is a problem that can happen during any phase of the sexual response cycle. SD prevents an individual from experiencing satisfaction from sexual activity. The sexual response cycle traditionally includes excitement, plateau, orgasm, and resolution. Desire and arousal are both parts of the excitement phase of the sexual response. Sexual dysfunction can affect a person at any age. Physical and/or medical conditions as well as psychological disorders can cause problems with sexual function. The side effects of some medications, including anti-depressants, can also affect sexual function.

Treatment-induced sexual dysfunction is an adverse effect associated with the administration of various anti-depressants. Tricyclic antidepressants, monoamine oxidase inhibitors, selective serotonin reuptake inhibitors (SSRIs), and serotonin-norepinephrine reuptake inhibitors (SNRIs) have all been linked to impairments in each phase of the sexual response cycle (interest, arousal, and orgasm) and to effects on sexual frequency and pleasure. Studies have shown that citalopram, escitalopram, fluoxetine, fluvoxamine, paroxetine, sertraline, duloxetine, venlafaxine, imipramine, and phenelzine were associated with significantly higher SD rates compared with placebo.

SD can be addressed by treating an underlying physical, medical, and/or psychological problem. Other methods for treating SD include medication (e.g., sildenafil (Viagra®), tadalafil (Cialis®), vardenafil (Levitra®, Staxyn®), avanafil (Stendra®), flibanserin (Addyi®) and bremelanotide (Vyleesi®)), use of mechanical aids (e.g., vacuum devices, penile implants), sex therapy, behavioral treatments, psychotherapy, and education and communication. Current methods used for treating or reducing treatment-induced SD include waiting for spontaneous remission or tolerance, dose reduction, drug holiday, non-pharmacologic interventions (e.g., exercise, psychotherapy), switching to a different antidepressant, and/or adding a medication to treat sexual adverse effects.

New and improved methods for treating SD, including treatment-induced SD are needed. Also of need are agents that prevent and treat CNS-relate diseases while exhibiting reduce or no side effects typically associated with antidepressants, particularly sexual dysfunction. Agents and methods using said agents described herein are directed toward this end.

### SUMMARY OF THE INVENTION

The present invention provides methods of treating sexual dysfunction in a patient in need thereof, comprising administering to the patient a therapeutically effective amount of a compound of the formula selected from the group consisting of:

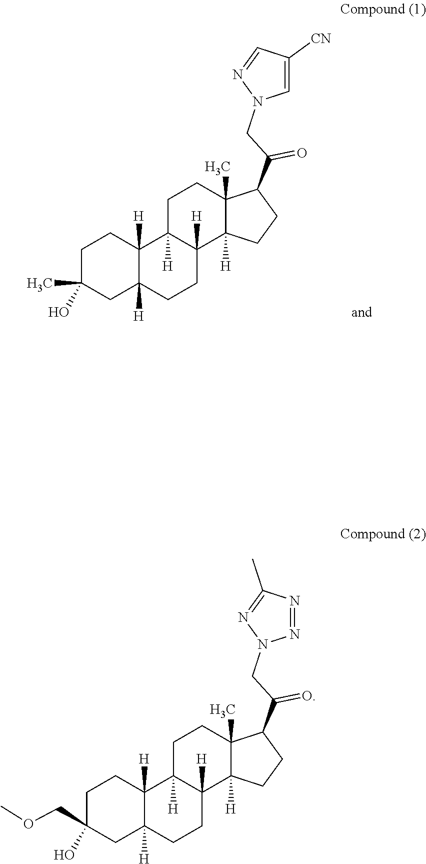

Another aspect of the invention provides methods of treating sexual dysfunction in a patient in need thereof, comprising administering to the patient a therapeutically effective amount of a pharmaceutically acceptable salt of a compound of the formula selected from the group consisting of:

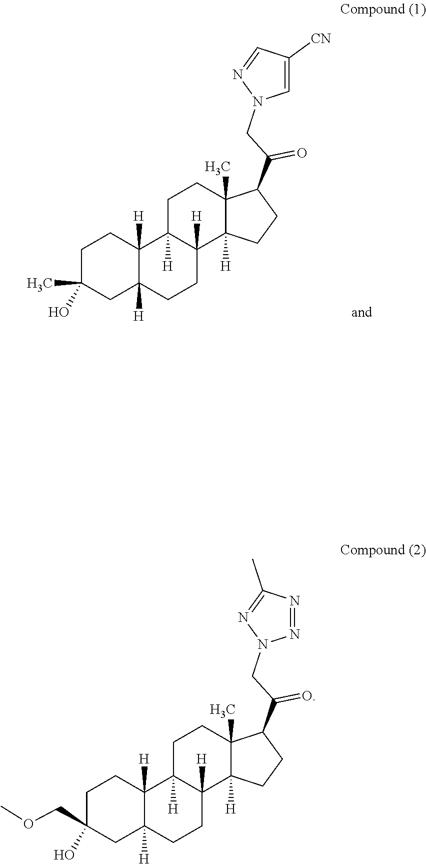

In some embodiments, the compound administered is a compound of the formula:

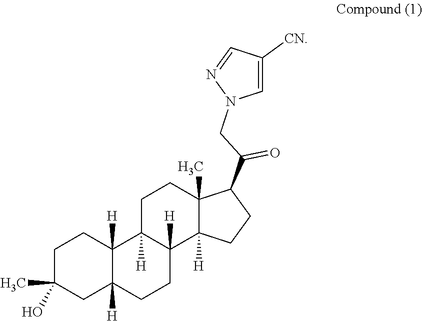

In some embodiments, the pharmaceutically acceptable salt administered is a pharmaceutically acceptable salt of a compound of the formula:

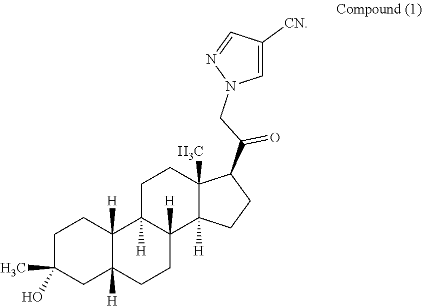

In some embodiments, the compound administered is a compound of the formula:

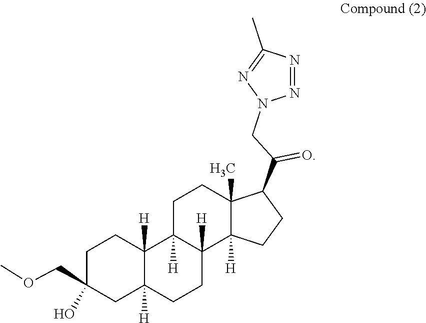

In some embodiments, the pharmaceutically acceptable salt administered is a pharmaceutically acceptable salt of a compound of the formula:

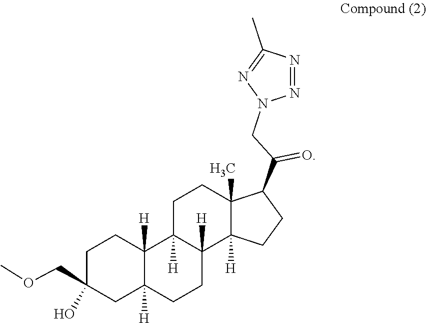

In some embodiments, the patient has (or suffers from) a CNS-related disorder. In some embodiments, the CNS-related disorder is selected from the group consisting of a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, and status epilepticus. In some embodiments, the CNS-related disorder is a mood disorder. In some embodiments, the mood disorder is major depressive disorder.

In some aspects, the compound is administered at a dose of about 20 mg to about 60 mg once a day. In some embodiments, the compound is administered at a dose of about 50 mg once a day for about 14 days. In some embodiments, the compound is administered at a dose of about 60 mg once a day for about 14 days.

In some aspects, the pharmaceutically acceptable salt of the compound is administered at a dose equivalent of about 20 mg to 60 mg of the free base compound once a day. In some embodiments, the pharmaceutically acceptable salt of the compound is administered at a dose equivalent of about 50 mg of the free base compound once a day for about 14 days. In some embodiments, the pharmaceutically acceptable salt of the compound is administered at a dose equivalent of about 60 mg of the free base compound once a day for about 14 days.

In some aspects, the sexual dysfunction is assessed by the Changes in Sexual Functioning Questionnaire-14. In some embodiments, the Changes in Sexual Functioning Questionnaire-14 score at day 42 is not greater than about the Changes in Sexual Functioning Questionnaire-14 score at baseline. In some embodiments, the patient is a female having a Changes in Sexual Functioning Questionnaire-14 score of no greater than 41 at 15 days, 28 days, or 42 days following the administration. In some embodiments, the patient is a male having a Changes in Sexual Functioning Questionnaire-14 score of no greater than 47 at 15 days, 28 days, or 42 days following the administration.

In some embodiments, the sexual dysfunction comprises decrease in or loss of one or more of sexual pleasure, sexual desire, sexual frequency, sexual interest, sexual arousal, sexual excitement, and orgasm.

Another aspect of the present invention provides methods of treating sexual dysfunction in a patient having major depressive disorder, the method comprising administering a therapeutically effective amount of a compound of the formula selected from the group consisting of:

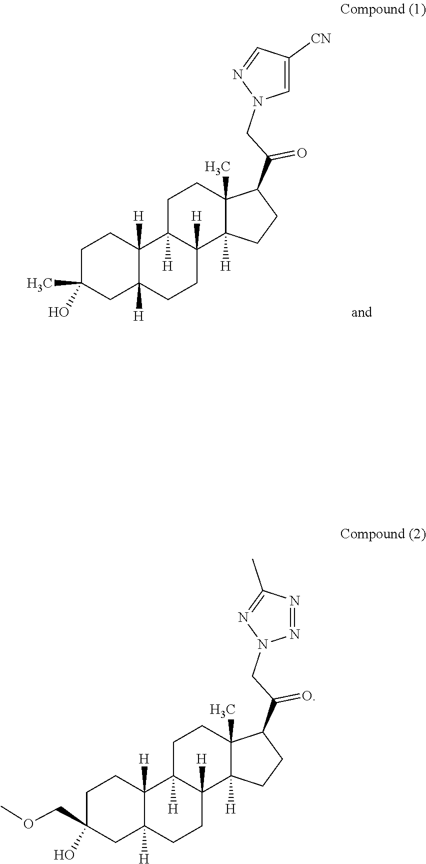

Another aspect of the present invention provides methods of treating sexual dysfunction in a patient having major depressive disorder, the method comprising administering a therapeutically effective amount of a pharmaceutically acceptable salt of a compound of the formula selected from the group consisting of:

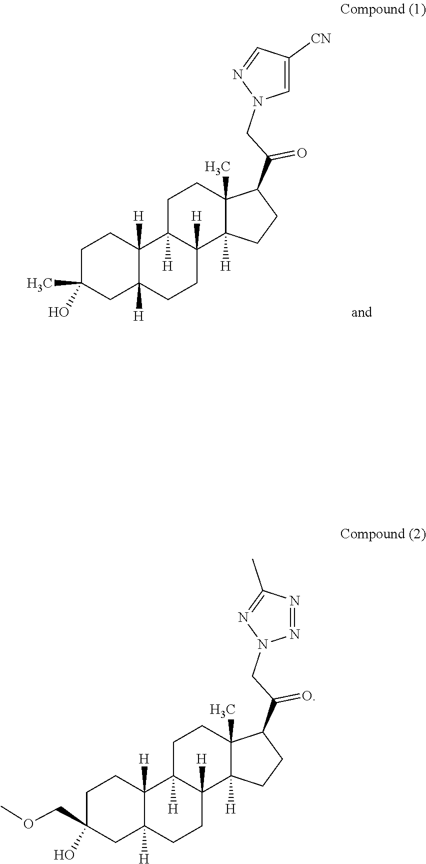

In some aspects, the compound administered is a compound of the formula:

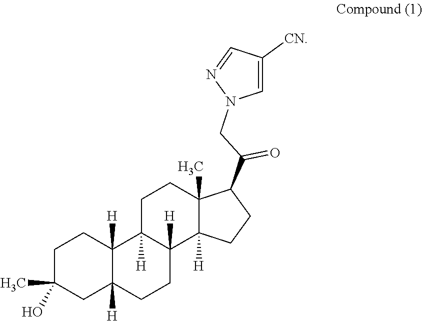

In some aspects, the pharmaceutically acceptable salt administered is a pharmaceutically acceptable salt of a compound of the formula:

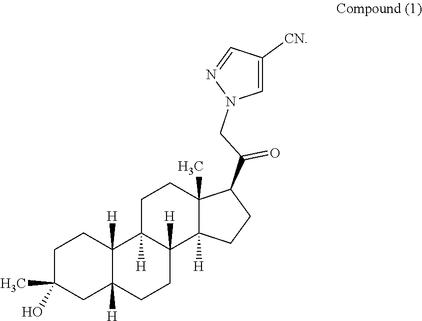

In some aspects, the compound administered is a compound of the formula:

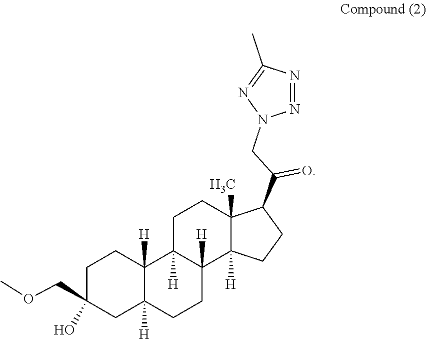

In some aspects, the pharmaceutically acceptable salt administered is a pharmaceutically acceptable salt of a compound of the formula:

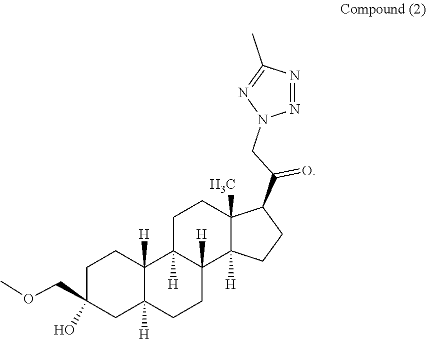

In some aspects, the compound is administered at a dose of about 20 mg to 60 mg once a day. In some embodiments, the compound is administered at a dose of about 50 mg once a day for about 14 days. In some embodiments, the compound is administered at a dose of about 60 mg once a day for about 14 days.

In some aspects, the pharmaceutically acceptable salt of the compound is administered at a dose equivalent of about 20 mg to about 60 mg of the free base compound once a day. In some embodiments, the pharmaceutically acceptable salt of the compound is administered at a dose equivalent of about 50 mg of the free base compound once a day for about 14 days. In some embodiments, the pharmaceutically acceptable salt of the compound is administered at a dose equivalent of about 60 mg of the free base compound once a day for about 14 days.

In some aspects, the sexual dysfunction is assessed by the Changes in Sexual Functioning Questionnaire-14. In some embodiments, the Changes in Sexual Functioning Questionnaire-14 score at day 42 is not greater than about the Changes in Sexual Functioning Questionnaire-14 score at baseline. In some embodiments, the patient is a female having a Changes in Sexual Functioning Questionnaire-14 score of no greater than 41 at 15 days, 28 days, or 42 days following the administration. In some embodiments, the patient is a male having a Changes in Sexual Functioning Questionnaire-14 score of no greater than 47 at 15 days, 28 days, or 42 days following the administration.

Another aspect of the present invention provides a method of maintaining sexual function in a patient who has a mood disorder, comprising administering to the patient a therapeutically effective amount of a compound of the formula selected from the group consisting of:

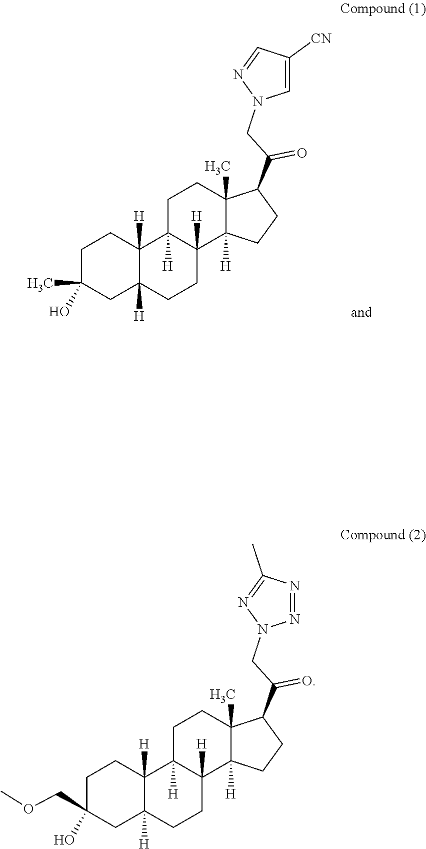

Another aspect of the present invention provides a method of maintaining sexual function in a patient who has a mood disorder, comprising administering to the patient a therapeutically effective amount of a pharmaceutically acceptable salt of a compound of the formula selected from the group consisting of:

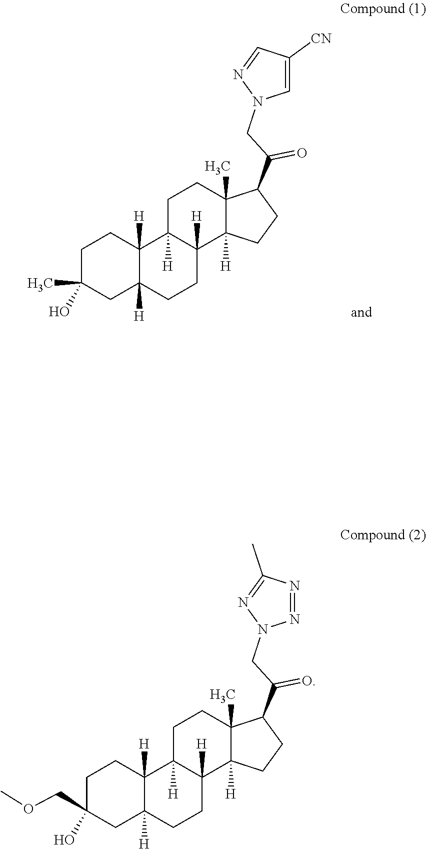

In some aspects, the compound administered is a compound of the formula:

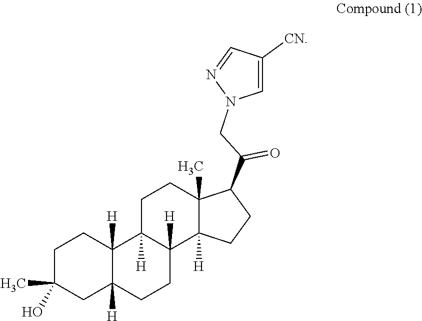

In some aspects, the pharmaceutically acceptable salt administered is a pharmaceutically acceptable salt of a compound of the formula:

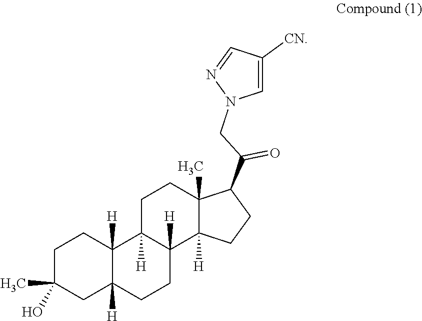

In some aspects, the compound administered is a compound of the formula:

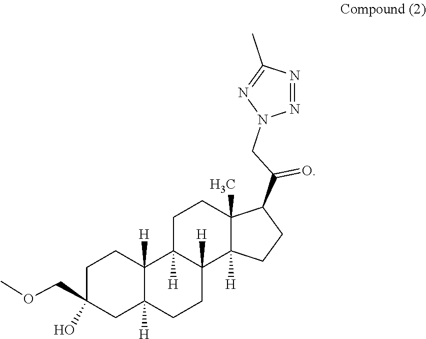

In some aspects, the pharmaceutically acceptable salt administered is a pharmaceutically acceptable salt of a compound of the formula:

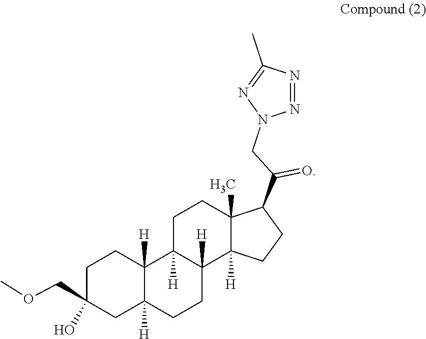

In some aspects, the mood disorder is major depressive disorder.

In some aspects, the compound is administered at a dose of about 20 mg to about 60 mg once a day. In some embodiments, the compound is administered at a dose of about 50 mg once a day for about 14 days. In some embodiments, the compound is administered at a dose of about 60 mg once a day for about 14 days.

In some aspects, the pharmaceutically acceptable salt of the compound is administered at a dose equivalent of about 20 mg to about 60 mg of the free base compound once a day. In some embodiments, the pharmaceutically acceptable salt of the compound is administered at a dose equivalent of about 50 mg of the free base compound once a day for about 14 days. In some embodiments, the pharmaceutically acceptable salt of the compound is administered at a dose equivalent of about 60 mg of the free base compound once a day for about 14 days.

In some aspects, the sexual dysfunction is assessed by the Changes in Sexual Functioning Questionnaire-14. In some embodiments, the Changes in Sexual Functioning Questionnaire-14 score at day 42 is not greater than about the Changes in Sexual Functioning Questionnaire-14 score at baseline. In some embodiments, the patient is a female having a Changes in Sexual Functioning Questionnaire-14 score of no greater than 41 at 15 days, 28 days, or 42 days following the administration. In some embodiments, the patient is a male having a Changes in Sexual Functioning Questionnaire-14 score of no greater than 47 at 15 days, 28 days, or 42 days following the administration.

Another aspect of the present invention provides a method of treating treatment-induced sexual dysfunction in a patient who is currently receiving or has received a course of chemical psychotherapy, comprising administering to the patient a therapeutically effective amount of a compound of the formula selected from the group consisting of:

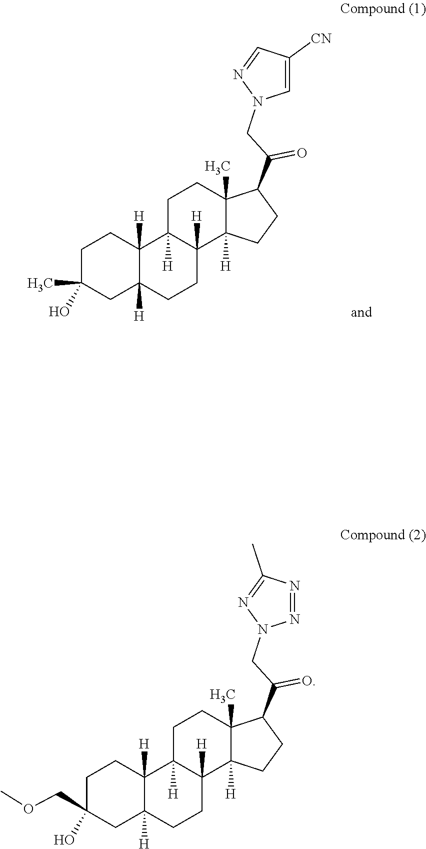

Another aspect of the present invention provides a method of treating treatment-induced sexual dysfunction in a patient who is currently receiving or has received a course of chemical psychotherapy, comprising administering to the patient a therapeutically effective amount of a pharmaceutically acceptable salt of a compound of the formula selected from the group consisting of:

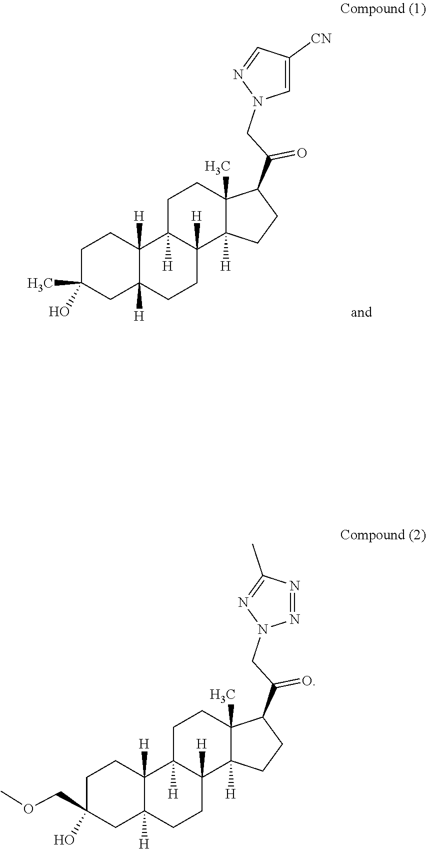

In some aspects, the compound administered is a compound of the formula:

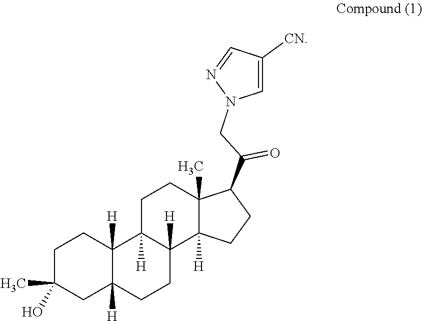

In some aspects, the pharmaceutically acceptable salt administered is a pharmaceutically acceptable salt of a compound of the formula:

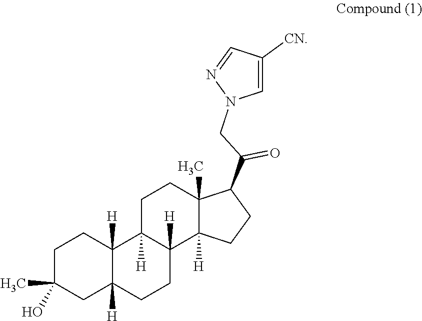

In some aspects, the compound administered is a compound of the formula:

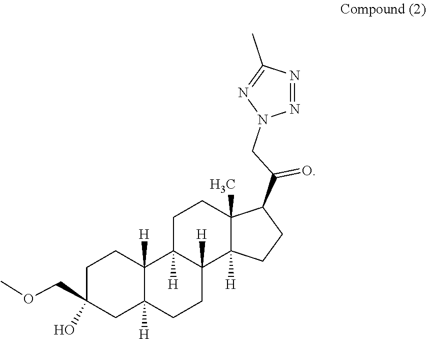

In some aspects, the pharmaceutically acceptable salt administered is a pharmaceutically acceptable salt of a compound of the formula:

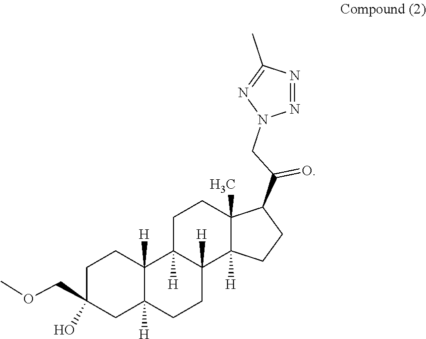

In some embodiments, the chemical psychotherapy is an antidepressant. In some embodiments, the antidepressant is selected from a selective serotonin reuptake inhibitor, a selective norepinephrine reuptake inhibitor, a tricyclic, a monoamine oxidase inhibitor, or an atypical antidepressant.

In some embodiments, the patient has (or suffers from) a CNS-related disorder. In some embodiments, the CNS-related disorder is selected from the group consisting of a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, and status epilepticus. In some embodiments, the CNS-related disorder is a mood disorder. In some embodiments, the mood disorder is major depressive disorder.

In some aspects, the compound is administered at a dose of about 20 mg to about 60 mg once a day. In some embodiments, the compound is administered at a dose of about 50 mg once a day for about 14 days. In some embodiments, the compound is administered at a dose of about 60 mg once a day for about 14 days.

In some aspects, the pharmaceutically acceptable salt of the compound is administered at a dose equivalent of about 20 mg to about 60 mg of the free base compound once a day. In some embodiments, the pharmaceutically acceptable salt of the compound is administered at a dose equivalent of about 50 mg of the free base compound once a day for about 14 days. In some embodiments, the pharmaceutically acceptable salt of the compound is administered at a dose equivalent of about 60 mg of the free base compound once a day for about 14 days.

In some aspects, the sexual dysfunction is assessed by the Changes in Sexual Functioning Questionnaire-14. In some embodiments, the Changes in Sexual Functioning Questionnaire-14 score at day 42 is not greater than about the Changes in Sexual Functioning Questionnaire-14 score at baseline. In some embodiments, the patient is a female having a Changes in Sexual Functioning Questionnaire-14 score of no greater than 41 at 15 days, 28 days, or 42 days following the administration. In some embodiments, the patient is a male having a Changes in Sexual Functioning Questionnaire-14 score of no greater than 47 at 15 days, 28 days, or 42 days following the administration.

### BRIEF DESCRIPTION OF THE FIGURES

FIG. 1 is a schematic showing the MOUNTAIN study design according to Example 1.

FIG. 2 is a graph showing mean Changes in Sexual Functioning Questionnaire-14 (CSFQ-14) total score change from baseline (±standard error) in patients who received 30 mg of Compound (1) or placebo at baseline and Days 15, 28, and 42 according to Example 1.

FIG. 3 is a graph showing percentage of patients with sexual dysfunction across treatment arms (Compound (1) vs placebo) at baseline and Days 15, 28, and 42 according to Example 1.

FIG. 4 is a graph showing female odds ratio at various CSFQ-14 subscales across treatment arms (Compound (1) vs placebo) at Days 15, 28, and 42 according to Example 1.

FIG. 5 is a graph showing male odds ratio at various CSFQ-14 subscales across treatment arms (Compound (1) vs placebo) at Days 15, 28, and 42 according to Example 1.

### DETAILED DESCRIPTION

The present invention generally relates to methods of treating sexual dysfunction and treatment-induced sexual dysfunction in a patient in need thereof by administering a compound of the formula selected from the group consisting of:

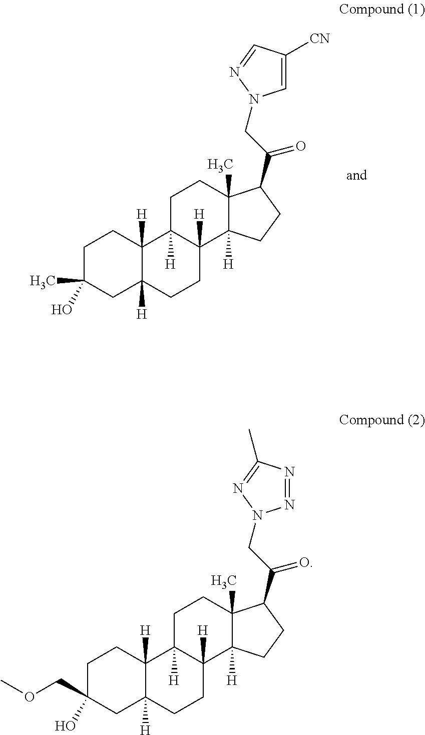

The present invention also relates to methods of treating sexual dysfunction and treatment-induced sexual dysfunction in a patient in need thereof by administering a pharmaceutically acceptable salt of a compound of the formula selected from the group consisting of:

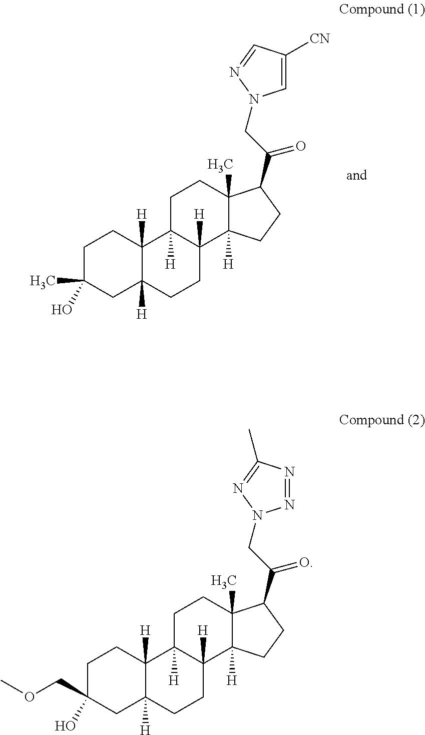

The present invention also relates to methods of treating of major depressive disorder in a patient in need thereof by administering to the patient a therapeutically effective amount of a compound of the formula selected from the group consisting of Compound (1) and Compound (2) wherein the patient maintains sexual function during the treatment:

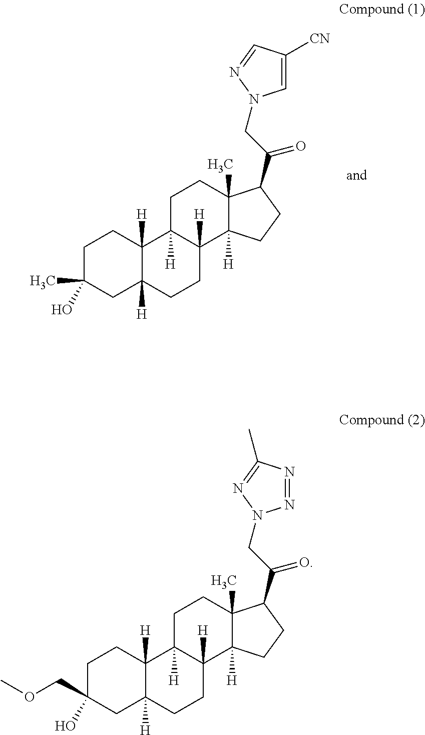

The present invention also relates to methods of treating of major depressive disorder in a patient in need thereof by administering to the patient a therapeutically effective amount of a pharmaceutically acceptable salt of a compound of the formula selected from the group consisting of Compound (1) and Compound (2), wherein the patient maintains sexual function during the treatment:

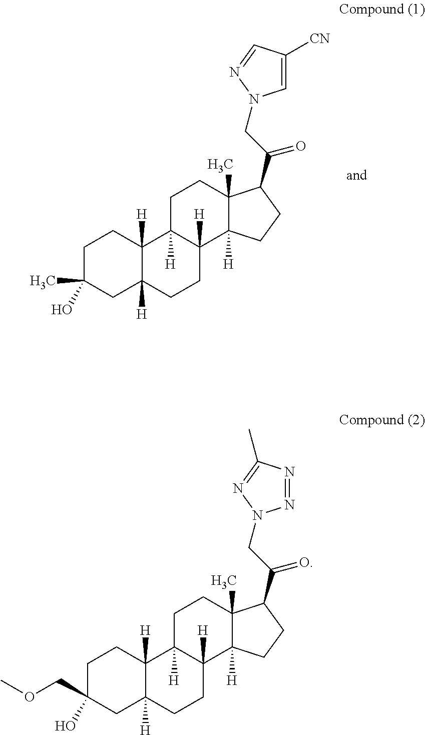

### I. Definitions

As used herein, “Compound (1)” refers to the compound having the formula (or structure):

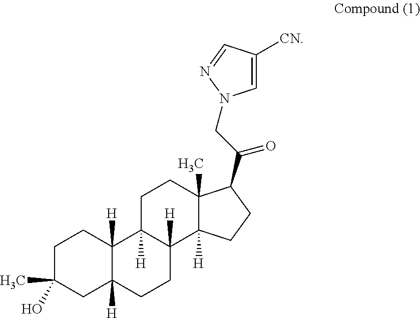

Compound (1) is also known as zuranolone, 3α-hydroxy-3β-methyl-21-(4-cyanopyrazol-1-yl)-5β-19-norpregnan-20-one, and by its IUPAC name: 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-1H-pyrazole-4-carbonitrile (CAS Registry Number 1632051-40-1). A method of chemically synthesizing Compound (1), was described in U.S. Pat. No. 9,512,165 and PCT Application Publication No. WO 2014/169833, each of which are incorporated by reference in their entirety herein. Several crystalline forms of Compound (1) and methods of preparing said forms were described in U.S. Patent Application Publication No. US 2019/0177359 and PCT Application Publication No. WO 2018/039378, each of which are incorporated by reference in their entirety herein.

As used herein, “Compound (2)” refers to the compound having the formula (or structure):

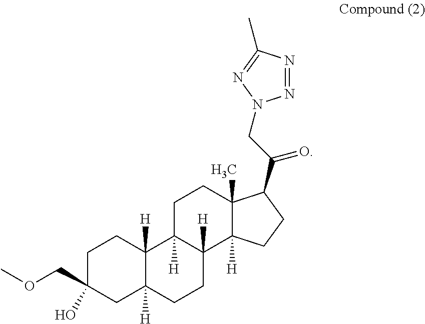

Compound (2) is also known as 3alpha-hydroxy-3beta-methoxymethyl-21-(5-methyl-2H-tetrazol-2-yl)-19-nor-5 alpha-pregnan-20-one, and by its IUPAC name: 1-((3R,5S,8R,9R,10S,13S,14S,17S)-3-hydroxy-3-(methoxymethyl)-13-methylhexadecahydro-1H-cyclopenta[a]phenanthren-17-yl)-2-(5-methyl-2H-tetrazol-2-yl)ethan-1-one (CAS Registry Number. 1832596-09-4). A method of chemically synthesizing Compound (2), was described in PCT Application Publication No. WO 2015/180679, which is incorporated by reference in its entirety herein.

Compound (1) and Compound (2) are neuroactive steroids that have been shown to be positive allosteric modulators of GABA_{A} receptors that target synaptic and extrasynaptic GABA_{A} receptors. As positive allosteric modulators of GABA_{A} receptors, Compound (1) and Compound (2) serve as therapeutic agents to treat CNS related disorders, e.g., depression, postpartum depression and major depressive disorder and to treat neurological conditions, e.g., essential tremor, epilepsy, and Parkinson's disease.

As used herein, the term “modulation” refers to the inhibition or potentiation of GABA_{A} receptor function. A “modulator” (e.g., a compound or pharmaceutically acceptable salt thereof that modulates GABA_{A} receptor function) may be, for example, an agonist, partial agonist, antagonist, or partial antagonist of the GABA_{A} receptor.

As used herein, and unless otherwise specified, the terms “treat,” “treating” and “treatment” contemplate an action that occurs while a subject is suffering from the specified disease, disorder or condition, which reduces the severity of the disease, disorder or condition (or any symptom thereof), or retards or slows the progression of the disease, disorder or condition (“therapeutic treatment”), and also contemplates a prophylactic action that occurs before a subject begins to suffer from the specified disease, disorder or condition.

The terms “disease”, “disorder”, and “condition” are used interchangeably herein.

As used herein, an “effective amount” of a compound (or pharmaceutically acceptable salt thereof) refers to an amount sufficient to elicit the desired biological response, e.g., to treat sexual dysfunction, e.g., to treat treatment-induced sexual dysfunction. As will be appreciated by those of ordinary skill in this art, the effective amount of a compound (or pharmaceutically acceptable salt thereof) of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound (or pharmaceutically acceptable salt thereof) is an amount sufficient to provide a therapeutic benefit in the treatment of a disease, disorder or condition, or to delay or minimize one or more symptoms associated with the disease, disorder or condition. A therapeutically effective amount of a compound (or pharmaceutically acceptable salt thereof) means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

A “patient” is a human (e.g., a male or female of any age group, e.g., a pediatric subject (e.g., infant, child, adolescent) or adult subject (e.g., young adult, middle-aged adult or senior adult)).

As used herein, the term “about”, when referring to a numerical value or range, allows for a degree of variability in the value or range, for example, within 10%, or within 5% of a stated value or of a stated limit of a range.

As used herein, the term “dose equivalent” means a bioequivalent dose. For example, the dose equivalent of a pharmaceutically acceptable salt of Compound (1) or Compound (2) for a 25 mg dose of Compound (1) or Compound (2) is the amount of the pharmaceutically acceptable salt (by weight) needed to provide a bioequivalent dose to the 25 mg dose of the free base of Compound (1) or Compound (2).

As used herein, the term “unit dosage form” is defined to refer to the form in which Compound (1) or Compound (2) is administered to the patient. Specifically, the unit dosage form can be, for example, a pill, capsule, or tablet. In some embodiments, the unit dosage form is a capsule. In some embodiments, the unit dosage form is a tablet.

As used herein, “solid dosage form” means a pharmaceutical dose(s) in solid form, e.g., tablets, capsules, granules, powders, sachets, reconstitutable powders, dry powder inhalers and chewables.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66 (1): 1-79.

“Sexual dysfunction” (or “SD”) can include but is not limited to decrease in or loss of sexual pleasure, sexual desire, sexual frequency, sexual interest, sexual arousal, sexual excitement, and orgasm, difficulty with erections, priapism, dysorgasmia, anorgasmia, dyspareunia, spontaneous orgasm, ejaculation disorders (delayed, inhibited, retrograde, spontaneous, decreased volume), sexual disinhibition, and sexual satisfaction.

“Sexual function” includes but is not limited to sexual pleasure, sexual desire, sexual frequency, sexual interest, sexual arousal, sexual excitement, and orgasm.

Sexual dysfunction is a problem that can occur during any phase of the sexual response cycle. SD prevents an individual from experiencing satisfaction from sexual activity. The sexual response cycle traditionally includes excitement, plateau, orgasm, and resolution. Desire and arousal are both parts of the excitement phase of the sexual response.

Sexual dysfunction is a prevalent symptom of major depressive disorder (MDD) and antidepressant therapy, with symptoms typically emerging from about 1 to about 3 weeks after treatment initiation. The estimated prevalence ranges from about 4% to about 73%, depending on the type of antidepressant administered.

### II. Methods of Treatment

Described herein are methods of treating sexual dysfunction and/or maintaining sexual function, in a patient, and/or methods of treating of major depressive disorder in a patient in need thereof with maintaining sexual function during the treatment, the method comprising administering to the patient a compound selected from the group consisting of Compound (1) and Compound (2); or a pharmaceutically acceptable salt thereof.

In one aspect, the present invention provides a method of treating sexual dysfunction in a patient in need thereof, the method comprising administering to the patient a therapeutically effective amount of a compound selected from the group consisting of Compound (1) and Compound (2).

In one aspect, the present invention provides a method of treating sexual dysfunction in a patient in need thereof, comprising administering to the patient a therapeutically effective amount of a pharmaceutically acceptable salt of a compound selected from the group consisting of Compound (1) and Compound (2).

In one aspect, the present invention provides a method of treating sexual dysfunction in a patient having a mood disorder, the method comprising administering a therapeutically effective amount of a compound selected from the group consisting of Compound (1) and Compound (2).

In another aspect, the present invention provides a method of treating sexual dysfunction in a patient having a mood disorder, the method comprising administering a therapeutically effective amount of a pharmaceutically acceptable salt of a compound selected from the group consisting of Compound (1) and Compound (2).

In some embodiments, the mood disorder is major depressive disorder.

In one aspect, the present invention provides a method of treating sexual dysfunction in a patient having major depressive disorder, the method comprising administering a therapeutically effective amount of a compound selected from the group consisting of Compound (1) and Compound (2).

In another aspect, the present invention provides a method of treating sexual dysfunction in a patient having major depressive disorder, the method comprising administering a therapeutically effective amount of a pharmaceutically acceptable salt of a compound selected from the group consisting of Compound (1) and Compound (2).

In one aspect, the present invention provides a method of treating major depressive disorder in a patient in need thereof, comprising administering to the patient a therapeutically effective amount of a compound selected from the group consisting of Compound (1) and Compound (2), wherein the patient maintains sexual function during the treatment.

In another aspect, the present invention provides a method of treating major depressive disorder in a patient in need thereof, comprising administering to the patient a therapeutically effective amount of a pharmaceutically acceptable salt of a compound selected from the group consisting of Compound (1) and Compound (2), wherein the patient maintains sexual function during the treatment.

In one aspect, the present invention provides a method of maintaining sexual function in a patient who has a mood disorder, comprising administering to the patient a therapeutically effective amount of a compound selected from the group consisting of Compound (1) and Compound (2).

In another aspect, the present invention provides a method of maintaining sexual function in a patient who has a mood disorder, comprising administering to the patient a therapeutically effective amount of a pharmaceutically acceptable salt of a compound selected from the group consisting of Compound (1) and Compound (2).

In some embodiments, the mood disorder is major depressive disorder.

In some embodiments, the compound administered is Compound (1).

In some embodiments, the pharmaceutically acceptable salt administered is a pharmaceutically acceptable salt of Compound (1).

In some embodiments, the compound administered is Compound (2).

In some embodiments, the pharmaceutically acceptable salt administered is a pharmaceutically acceptable salt of Compound (2).

In some embodiments, sexual function includes sexual pleasure, sexual desire, sexual frequency, sexual interest, sexual arousal, sexual excitement, or orgasm.

In some embodiments, maintaining sexual function during the treatment refers to the patient having no impairment of sexual function during treatment. In some embodiments, maintaining sexual function during the treatment refers to the patient having no significant differences in sexual function during treatment. In some embodiments, maintaining sexual function during the treatment refers to the patient having no treatment-emergent sexual dysfunction. In some embodiments, the patient maintains sexual function during the treatment regardless of sex, e.g.., female or male patient.

### Sexual Dysfunction Assessment

Sexual dysfunction (SD) can be assessed by the 14-item Changes in Sexual Functioning Questionnaire (CSFQ-14), The CSFQ-14 yields scores for three scales corresponding to the phases of the sexual response cycle (e.g., desire, arousal, and orgasm). The CSFQ-14 has 5 sub-scales that measure pleasure, desire/frequency, desire/interest, arousal/excitement, and orgasm/completion. In some embodiments, the CSFQ-14 total scores are assessed at baseline, Day 15, Day 28, and/or Day 42 after commencement of a trial.

In some embodiments, a CSFQ-14 total score of less than or equal to 41 in female patients indicates sexual dysfunction.

In some embodiments, a CSFQ-14 total score of less than or equal to 47 in male patients indicates sexual dysfunction.

In certain embodiments, the CSFQ-14 total score at day 42 is not greater than about the CSFQ-14 total score at baseline. In some embodiments, the patient is a female and the patient has a CSFQ-14 total score of no more than 41 at 15 days following the administration. In some embodiments, the patient is a female and the patient has a CSFQ-14 total score of no more than 41 at 28 days following the administration. In some embodiments, the patient is a female and the patient has a CSFQ-14 total score of no more than 41 at 42 days following the administration. In some embodiments, the patient is a female and the patient has a CSFQ-14 total score of no more than 41 at 15 days, 28 days, or 42 days following the administration. In some embodiments, the patient is a male and the patient has a CSFQ-14 total score of no more than 47 at 15 days following the administration. In some embodiments, the patient is a male and the patient has a CSFQ-14 total score of no more than 47 at 28 days following the administration. In some embodiments, the patient is a male and the patient has a CSFQ-14 total score of no more than 47 at 42 days following the administration. In some embodiments, the patient is a male and the patient has a CSFQ-14 total score of no more than 47 at 15 days, 28 days, or 42 days following the administration.

In some embodiments, the methods described herein provide a therapeutic effect as measured by an increase in CSFQ-14 total score. In some embodiments, the therapeutic effect is an increase from baseline in CSFQ-14 total score after beginning administration of Compound (1) or Compound (2). In some embodiments, the therapeutic effect is an increase from baseline in CSFQ-14 total score of about 2.5 to about 5.0, or about 3.0 to about 4.0, or about 3.0 to about 3.5, after administration of Compound (1) or Compound (2) in female patients. In some embodiments, the therapeutic effect is an increase from baseline in CSFQ-14 total score of about 1.0 to about 5.0, or about 1.5 to about 3.5, or about 2.0 to about 3.5, after administration of Compound (1) or Compound (2) in male patients.

In some embodiments, the methods described herein provide a therapeutic effect (e.g., as measured by an increase in CSFQ-14 total score) within about 42, about 28, or about 15 days. In some embodiments, the therapeutic effect is an increase from baseline in CSFQ-14 total score at the end of a treatment period (e.g., about 42, about 28, or about 15 days after beginning administration of Compound (1) or Compound (2)). In some embodiments, the therapeutic effect is an increase from baseline in CSFQ-14 total score of about 2.5 to about 5.0, or about 3.0 to about 4.0, or about 3.0 to about 3.5 at the end of a treatment period (e.g., about 42, about 28, or about 15 days after beginning administration of Compound (1) or Compound (2)) in female patients. In some embodiments, the therapeutic effect is an increase from baseline in CSFQ-14 total score of about 1.0 to about 5.0, or about 1.5 to about 3,5, or about 2.0 to about 3.5, at the end of a treatment period (e.g., about 42, about 28, or about 15 days after beginning administration of Compound (1) or Compound (2)) in male patients.

In some embodiments, the therapeutic effect is an increase from baseline in CSFQ-14 total score after administration of a pharmaceutically acceptable salt of Compound (1) or Compound (2). In some embodiments, the therapeutic effect is an increase from baseline in CSFQ-14 total score of about 2.5 to about 5.0, or about 3.0 to about 4.0, or about 3.0 to about 3.5, after administration of a pharmaceutically acceptable salt of Compound (1) or Compound (2) in female patients. In some embodiments, the therapeutic effect is an increase from baseline in CSFQ-14 total score of about 1.0 to about 5.0, or about 1.5 to about 3.5, or about 2.0 to about 3.5, after administration of a pharmaceutically acceptable salt of Compound (1) or Compound (2) in male patients.

In some embodiments, the therapeutic effect is an increase from baseline in CSFQ-14 total score at the end of a treatment period (e.g., about 42, about 28, or about 15 days after beginning administration of a pharmaceutically acceptable salt of Compound (1) or Compound (2)). In some embodiments, the therapeutic effect is an increase from baseline in CSFQ-14 total score of about 2.5 to about 5.0, or about 3.0 to about 4.0, or about 3.0 to about 3.5 at the end of a treatment period (e.g., about 42, about 28, or about 15 days after beginning administration of a pharmaceutically acceptable salt of Compound (1) or Compound (2)) in female patients. In some embodiments, the therapeutic effect is an increase from baseline in CSFQ-14 total score of about 1.0 to about 5.0, or about 1.5 to about 3.5, or about 2.0 to about 3.5, at the end of a treatment period (e.g., about 42, about 28, or about 15 days after beginning administration of a pharmaceutically acceptable salt of Compound (1) or Compound (2)) in male patients.

In some embodiments, the methods described herein provide a therapeutic effect or a prophylactic effect as measured by a CSFQ-14 total score, whereby the patient treated with Compound (1) or Compound (2) has the same, similar, or no significantly different CSFQ-14 total score compared to a patient that has not been treated with Compound (1) or Compound (2). In some embodiments, the methods described herein provide a therapeutic effect or a prophylactic effect as measured by a CSFQ-14 total score, whereby the patient treated with a pharmaceutically acceptable salt of Compound (1) or Compound (2) has the same, similar (within about +10%), or no significantly different (e.g., as measured by p-values) CSFQ-14 total score compared to a patient that has not been treated with a pharmaceutically acceptable salt of Compound (1) or Compound (2).

### CNS-Related Disorders

In some embodiments, the patient has (or suffers from) a CNS-related disorder. Exemplary CNS conditions include, but are not limited to, sleep disorders [e.g., insomnia], mood disorders [e.g., depression (e.g., major depressive disorder (MDD) or postpartum depression (PPD)), dysthymic disorder (e.g., mild depression), bipolar disorder (e.g., I and/or II), anxiety disorders (e.g., generalized anxiety disorder (GAD), social anxiety disorder), stress, post-traumatic stress disorder (PTSD), compulsive disorders (e.g., obsessive compulsive disorder (OCD))], schizophrenia spectrum disorders [e.g., schizophrenia, schizoaffective disorder], convulsive disorders [e.g., epilepsy (e.g., status epilepticus (SE)), seizures], disorders of memory and/or cognition [e.g., attention disorders (e.g., attention deficit hyperactivity disorder (ADHD)), dementia (e.g., Alzheimer's type dementia, Lewis body type dementia, vascular type dementia], movement disorders [e.g., Huntington's disease, Parkinson's disease], personality disorders [e.g., anti-social personality disorder, obsessive compulsive personality disorder], autism spectrum disorders (ASD) [e.g., autism, monogenetic causes of autism such as synaptophathy's, e.g., Rett syndrome, Fragile X syndrome, Angelman syndrome], pain [e.g., neuropathic pain, injury related pain syndromes, acute pain, chronic pain], traumatic brain injury (TBI), vascular diseases [e.g., stroke, ischemia, vascular malformations], substance abuse disorders and/or withdrawal syndromes [e.g., addition to opiates, cocaine, and/or alcohol], and tinnitus.

In certain embodiments, the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, or status epilepticus. In certain embodiments, the CNS-related disorder is depression. In certain embodiments, the CNS-related disorder is a mood disorder. In certain embodiments, the CNS-related disorder is major depressive disorder. In certain embodiments, the major depressive disorder is moderate major depressive disorder. In certain embodiments, the major depressive disorder is severe major depressive disorder.

### Mood Disorders

A mood disorder is, for example, clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior.

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

In certain embodiments, the patient has a major depressive disorder. In some embodiments, the major depressive disorder is moderate major depressive disorder. In certain embodiments, the major depressive disorder is severe major depressive disorder.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postnatal depression (PND) is also referred to as postpartum depression (PPD), and refers to a type of clinical depression that affects women after childbirth. Symptoms can include sadness, fatigue, changes in sleeping and eating habits, reduced sexual desire, crying episodes, anxiety, and irritability.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent felling of worthlessness or hopelessness.

Mood disorder associated with conditions or disorders of women's health refers to mood disorders (e.g., depression) associated with (e.g., resulting from) a condition or disorder of women's health.

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

Depression and its associated behavioral patterns may contribute to other disease processes, such as metabolic syndrome, that can exacerbate female sexual dysfunction. Adverse sexual effects in some women taking antidepressants can include problems with sexual desire and sexual arousal.

Known depression scales, e.g., the Hamilton Depression Rating Scale (HAM-D or HAMD-17), the Clinical Global Impression-Improvement Scale (CGI), and the Montgomery-Asberg Depression Rating Scale (MADRS) can be used to identify patients that have a major depressive disorder.

In some embodiments, the HAM-D total score of the patient before treatment with Compound (1) or Compound (2) is at least 24. In some embodiments, the HAM-D total score of the patient before treatment with Compound (1) or Compound (2) is at least 22. In some embodiments, the HAM-D total score of the patient before treatment with Compound (1) or Compound (2) is at least 20. In some embodiments, the HAM-D total score of the patient before treatment with Compound (1) or Compound (2) is between and including 14 and 18. In some embodiments, the MADRS total score of the patient before treatment with Compound (1) or Compound (2) is at least 32. In some embodiments, the MADRS total score of the patient before treatment with Compound (1) or Compound (2) is at least 28.

Another aspect of the invention provides a method of treating treatment-induced sexual function in a patient who is currently receiving or has received a course of chemical psychotherapy, comprising administering to the patient a therapeutically effective amount of a compound selected from the group consisting of Compound (1) and Compound (2).

In a separate aspect, the invention provides a method of treating treatment-induced sexual function in a patient who is currently receiving or has received a course of chemical psychotherapy, comprising administering to the patient a therapeutically effective amount of a pharmaceutically acceptable salt of a compound selected from the group consisting of Compound (1) and Compound (2).

In some embodiments, the compound administered is Compound (1). In some embodiments, the pharmaceutically acceptable salt administered is a pharmaceutically acceptable salt of Compound (1).

In some embodiments, the compound administered is Compound (2). In some embodiments, the pharmaceutically acceptable salt administered is a pharmaceutically acceptable salt of Compound (2).

“Treatment-induced sexual dysfunction” or “Treatment-emergent sexual dysfunction” refers to sexual dysfunction resulting from receiving medications for the treatment of CNS-related disorders. Tricyclic antidepressants, monoamine oxidase inhibitors, selective serotonin reuptake inhibitors (SSRIs), and serotonin-norepinephrine reuptake inhibitors (SNRIs) have all been linked to treatment-induced sexual dysfunction.

In some embodiments, the chemical psychotherapy is an antidepressant. In some embodiments, the antidepressant is selected from a selective serotonin reuptake inhibitor (SSRI), a selective norepinephrine reuptake inhibitor (NERI), a tricyclic, a monoamine oxidase inhibitor, or an atypical antidepressant.

SSRIs include antidepressants that increase the level of serotonin in the brain. Exemplary SSRIs include, but are not limited to, Citalopram (Celexa), Escitalopram (Lexapro), Fluoxetine (Prozac), Fluvoxamine (Luvox), Paroxetine (Paxil), and Sertraline (Zoloft).

Exemplary NERIs include, but are not limited to, Atomoxetine (Strattera), Reboxetine (Edronax, Vestra), Bupropion (Wellbutrin, Zyban), Duloxetine, Desipramine (Norpramin), Amedalin (UK-3540-1), Daledalin (UK-3557-15), Edivoxetine (LY-2216684), Esreboxetine, Lortalamine (LM-1404), Nisoxetine (LY-94,939), Talopram (tasulopram) (Lu 3-010), Talsupram (Lu 5-005), Tandamine (AY-23,946), and Viloxazine (Vivalan).

Exemplary tricyclic antidepressants include, but are not limited to, Amitriptyline (Elavil, Endep), Amitriptylinoxide (Amioxid, Ambivalon, Equilibrin), Clomipramine (Anafranil), Desipramine (Norpramin, Pertofrane), Dibenzepin (Noveril, Victoril), Dimetacrine (Istonil), Dosulepin (Prothiaden), Doxepin (Adapin, Sinequan), Imipramine (Tofranil), Lofepramine (Lomont, Gamanil), Melitracen (Dixeran, Melixeran, Trausabun), Nitroxazepine (Sintamil), Nortriptyline (Pamelor, Aventyl), Noxiptiline (Agedal, Elronon, Nogedal), Opipramol (Insidon), Pipofezine (Azafen/Azaphen), Protriptyline (Vivactil), and Trimipramine (Surmontil).

Exemplary monoamine oxidase inhibitors include, but are not limited to, Isocarboxazid (Marplan), Phenelzine (Nardil), Tranylcypromine (Parnate), Selegiline (Eldepryl, Zelapar, Emsam), Metralindole (Inkazan), Moclobemide (Aurorix, Manerix), Pirlindole (Pirazidol), Toloxatone (Humoryl), and Bifemelane (Alnert, Celeport).

### Dosage

In some embodiments, Compound (1) or Compound (2) is administered at a dose of about 10 mg to about 100 mg. In some embodiments, Compound (1) or Compound (2) is administered at a dose of about 15 mg to about 75 mg. In some embodiments, Compound (1) or Compound (2) is administered at a dose of about 20 mg to about 60 mg. In some embodiments, Compound (1) or Compound (2) is administered at a dose of about 20 mg, about 25 mg, about 30 mg, about 35 mg, about 40 mg, about 45 mg, about 50 mg, about 55 mg, or about 60 mg. In some embodiments, Compound (1) or Compound (2) is administered at a dose of about 50 mg. In some embodiments, Compound (1) or Compound (2) is administered at a dose of about 60 mg.

In some embodiments, Compound (1) is administered at a dose of about 10 mg to about 100 mg. In some embodiments, Compound (1) is administered at a dose of about 15 mg to about 75 mg. In some embodiments, Compound (1) is administered at a dose of about 20 mg to about 60 mg. In some embodiments, Compound (1) is administered at a dose of about 20 mg, about 25 mg, about 30 mg, about 35 mg, about 40 mg, about 45 mg, about 50 mg, about 55 mg, or about 60 mg. In some embodiments, Compound (1) is administered at a dose of about 50 mg. In some embodiments, Compound (1) is administered at a dose of about 60 mg.

In some embodiments, Compound (2) is administered at a dose of about 10 mg to about 100 mg. In some embodiments, Compound (2) is administered at a dose of about 15 mg to about 75 mg. In some embodiments, Compound (2) is administered at a dose of about 20 mg to about 60 mg. In some embodiments, Compound (2) is administered at a dose of about 20 mg, about 25 mg, about 30 mg, about 35 mg, about 40 mg, about 45 mg, about 50 mg, about 55 mg, or about 60 mg. In some embodiments, Compound (2) is administered at a dose of about 50 mg. In some embodiments, Compound (2) is administered at a dose of about 60 mg.

In some embodiments, Compound (1) is administered at a dose of about 50 mg.

In some embodiments, Compound (2) is administered at a dose of about 60 mg.

In some embodiments, Compound (1) or Compound (2) is administered at a dose of about 10 mg to about 100 mg once a day. In some embodiments, Compound (1) or Compound (2) is administered at a dose of about 20 mg to about 60 mg once a day. In some embodiments, Compound (1) or Compound (2) is administered at a dose of about 50 mg once a day. In some embodiments, Compound (1) or Compound (2) is administered at a dose of about 60 mg once a day. For example, Compound (1) is administered at a dose of about 10 mg to about 100 mg once a day. In other examples, Compound (1) is administered at a dose of about 20 mg to about 60 mg once a day. In some examples, Compound (1) is administered at a dose of about 50 mg once a day. And, in other examples, Compound (1) is administered at a dose of about 60 mg once a day. In still other examples, Compound (2) is administered at a dose of about 10 mg to about 100 mg once a day. In other examples, Compound (2) is administered at a dose of about 20 mg to about 60 mg once a day. In some examples, Compound (2) is administered at a dose of about 50 mg once a day. And, in other examples, Compound (2) is administered at a dose of about 60 mg once a day.

In some embodiments, Compound (1) or Compound (2) is administered at a dose of about 50 mg or about 60 mg once a day for less than 2 weeks. In some embodiments, Compound (1) or Compound (2) is administered at a dose of about 50 mg or about 60 mg once a day for 1 day. In some embodiments, Compound (1) or Compound (2) is administered at a dose of about 50 mg or about 60 mg once a day for 2 days. In some embodiments, Compound (1) is administered at a dose of about 50 mg once a day for about 14 days. In some embodiments, Compound (1) or Compound (2) is administered at a dose of about 50 mg or about 60 mg once a day for about 28 days. In some embodiments, Compound (1) or Compound (2) is administered at a dose of about 50 mg or about 60 mg once a day for about 42 days. In some embodiments, Compound (1) or Compound (2) is administered at a dose of about 50 mg or about 60 mg once a day for at least 6 months. In some embodiments, Compound (1) or Compound (2) is administered at a dose of about 50 mg or about 60 mg once a day for at least 1 year. In some embodiments, Compound (1) or Compound (2) is administered at a dose of about 50 mg or about 60 mg once a day for life.

In some embodiments, Compound (1) is administered at a dose of about 50 mg or about 60 mg once a day for less than 2 weeks. In some embodiments, Compound (1) is administered at a dose of about 50 mg or about 60 mg once a day for 1 day. In some embodiments, Compound (1) is administered at a dose of about 50 mg or about 60 mg once a day for 2 days. In some embodiments, Compound (1) is administered at a dose of about 50 mg once a day for about 14 days. In some embodiments, Compound (1) is administered at a dose of about 50 mg or about 60 mg once a day for about 28 days. In some embodiments, Compound (1) is administered at a dose of about 50 mg or about 60 mg once a day for about 42 days. In some embodiments, Compound (1) is administered at a dose of about 50 mg or about 60 mg once a day for at least 6 months. In some embodiments, Compound (1) is administered at a dose of about 50 mg or about 60 mg once a day for at least 1 year. In some embodiments, Compound (1) is administered at a dose of about 50 mg or about 60 mg once a day for life.

In some embodiments, Compound (2) is administered at a dose of about 50 mg or about 60 mg once a day for less than 2 weeks. In some embodiments, Compound (2) is administered at a dose of about 50 mg or about 60 mg once a day for 1 day. In some embodiments, Compound (2) is administered at a dose of about 50 mg or about 60 mg once a day for 2 days. In some embodiments, Compound (2) is administered at a dose of about 50 mg once a day for about 14 days. In some embodiments, Compound (2) is administered at a dose of about 50 mg or about 60 mg once a day for about 28 days. In some embodiments, Compound (2) is administered at a dose of about 50 mg or about 60 mg once a day for about 42 days. In some embodiments, Compound (2) is administered at a dose of about 50 mg or about 60 mg once a day for at least 6 months. In some embodiments, Compound (2) is administered at a dose of about 50 mg or about 60 mg once a day for at least 1 year. In some embodiments, Compound (2) is administered at a dose of about 50 mg or about 60 mg once a day for life.

In some embodiments, the patient is administered Compound (1) or Compound (2) at night. In some embodiments, the patient is administered Compound (1) or Compound (2) no later than 1 hour before the patient sleeps. In some embodiments, the patient is administered Compound (1) or Compound (2) no later than 15 minutes before the patient sleeps. In some embodiments, Compound (1) or Compound (2) is administered chronically.

In some embodiments, the patient is administered Compound (1) at night. In some embodiments, the patient is administered Compound (1) no later than 1 hour before the patient sleeps. In some embodiments, the patient is administered Compound (1) no later than 15 minutes before the patient sleeps. In some embodiments, Compound (1) is administered chronically.

In some embodiments, the patient is administered Compound (2) at night. In some embodiments, the patient is administered Compound (2) no later than 1 hour before the patient sleeps. In some embodiments, the patient is administered Compound (2) no later than 15 minutes before the patient sleeps. In some embodiments, Compound (2) is administered chronically.

In other embodiments, the pharmaceutically acceptable salt of Compound (1) or Compound (2) is administered at a dose equivalent of about 10 mg to about 100 mg of the free base compound. In some embodiments, the pharmaceutically acceptable salt of Compound (1) or Compound (2) is administered at a dose equivalent of about 15 mg to about 75 mg of the free base compound. In some embodiments, the pharmaceutically acceptable salt of Compound (1) or Compound (2) is administered at a dose equivalent of about 20 mg to about 60 mg of the free base compound. In some embodiments, the pharmaceutically acceptable salt of Compound (1) or Compound (2) is administered at a dose equivalent of about 20 mg, about 25 mg, about 30 mg, about 35 mg, about 40 mg, about 45 mg, about 50 mg, about 55 mg, or about 60 mg of the free base compound. In some embodiments, the pharmaceutically acceptable salt of Compound (1) or Compound (2) is administered at a dose equivalent of about 50 mg of the free base compound. In some embodiments, the pharmaceutically acceptable salt of Compound (1) or Compound (2) is administered at a dose equivalent of about 60 mg of the free base compound.

In some embodiments, the pharmaceutically acceptable salt of Compound (1) is administered at a dose equivalent of about 50 mg of the free base compound.

In some embodiments, the pharmaceutically acceptable salt of Compound (2) is administered at a dose equivalent of about 60 mg of the free base compound.

In some embodiments, the pharmaceutically acceptable salt of Compound (1) or Compound (2) is administered at a dose equivalent of about 10 mg to about 100 mg of the free base compound once a day. In some embodiments, the pharmaceutically acceptable salt of Compound (1) or Compound (2) is administered at a dose equivalent of about 20 mg to about 60 mg of the free base compound once a day. In some embodiments, the pharmaceutically acceptable salt of Compound (1) or Compound (2) is administered at a dose equivalent of about 20 mg, about 25 mg, about 30 mg, about 35 mg, about 40 mg, about 45 mg, about 50 mg, about 55 mg, or about 60 mg of the free base compound once a day. In some embodiments, the pharmaceutically acceptable salt of Compound (1) or Compound (2) is administered at a dose equivalent of about 50 mg of the free base compound once a day. In some embodiments, the pharmaceutically acceptable salt of Compound (1) or Compound (2) is administered at a dose equivalent of about 60 mg of the free base compound once a day.

In some embodiments, the pharmaceutically acceptable salt of Compound (1) or Compound (2) is administered at a dose equivalent of about 50 mg or about 60 mg of the free base compound once a day for less than 2 weeks. In some embodiments, the pharmaceutically acceptable salt of Compound (1) or Compound (2) is administered at a dose equivalent of about 50 mg or about 60 mg of the free base compound once a day for 1 day. In some embodiments, the pharmaceutically acceptable salt of Compound (1) or Compound (2) is administered at a dose equivalent of about 50 mg or about 60 mg of the free base compound once a day for 2 days. In some embodiments, the pharmaceutically acceptable salt of Compound (1) or Compound (2) is administered at a dose equivalent of about 50 mg or about 60 mg of the free base compound once a day for about 14 days. In some embodiments, the pharmaceutically acceptable salt of Compound (1) or Compound (2) is administered at a dose equivalent of about 50 mg or about 60 mg of the free base compound once a day for about 28 days. In some embodiments, the pharmaceutically acceptable salt of Compound (1) or Compound (2) is administered at a dose equivalent of about 50 mg or about 60 mg of the free base compound once a day for about 42 days. In some embodiments, the pharmaceutically acceptable salt of Compound (1) or Compound (2) is administered at a dose equivalent of about 50 mg or about 60 mg of the free base compound once a day for at least 6 months. In some embodiments, the pharmaceutically acceptable salt of Compound (1) or Compound (2) is administered at a dose equivalent of about 50 mg or about 60 mg of the free base compound once a day for at least 1 year. In some embodiments, the pharmaceutically acceptable salt of Compound (1) or Compound (2) is administered at a dose equivalent of about 50 mg or about 60 mg of the free base compound once a day for life.

In some embodiments, the pharmaceutically acceptable salt of Compound (1) or Compound (2) is administered at night. In some embodiments, the pharmaceutically acceptable salt of Compound (1) or Compound (2) is administered no later than 1 hour before the patient sleeps. In some embodiments, the pharmaceutically acceptable salt of Compound (1) or Compound (2) is administered no later than 15 minutes before the patient sleeps. In some embodiments, the pharmaceutically acceptable salt of Compound (1) or Compound (2) is administered chronically.

### III. Pharmaceutical Compositions

Another aspect of the disclosure provides a pharmaceutical composition comprising Compound (1) or Compound (2) (also referred to as the “active ingredient(s)”), and a pharmaceutically acceptable excipient for use in treating sexual dysfunction and/or maintaining sexual function in a patient. In another aspect, the disclosure provides a pharmaceutical composition comprising a pharmaceutically acceptable salt of the active ingredient and a pharmaceutically acceptable excipient for use in treating sexual dysfunction and/or maintaining sexual function in a patient. In certain embodiments, the pharmaceutical composition comprises an effective amount of the active ingredient or a pharmaceutically acceptable salt of the active ingredient. In certain embodiments, the pharmaceutical composition comprises a therapeutically effective amount of the active ingredient or a pharmaceutically acceptable salt of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In some embodiments, the pharmaceutical composition is administered orally.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

The above-described components for orally administrable, injectable or topically administrable compositions are merely representative. Other materials, as well as processing techniques and the like, are set forth in Part 8 of Remington's Pharmaceutical Sciences, 17th edition, 1985, Mack Publishing Company, Easton, Pennsylvania, which is incorporated herein by reference.

The compositions of the present invention can also be administered in sustained release forms or from sustained release drug delivery systems. A description of representative sustained release materials can be found in Remington's Pharmaceutical Sciences.

Compound (1) or Compound (2), or a pharmaceutically acceptable salt thereof, can be administered as the sole active agent, or they can be administered in combination with other active agents. In one aspect, the present invention provides a combination Compound (1) or Compound (2), or a pharmaceutically acceptable salt thereof, and another pharmacologically active agent. Administration in combination can proceed by any technique apparent to those of skill in the art including, for example, separate, sequential, concurrent, and alternating administration.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21st ed., Lippincott Williams & Wilkins, 2005.

### IV. Examples

In order that the invention described herein may be more fully understood, the following examples are set forth. The examples described in this application are offered to illustrate the crystalline solid forms provided herein and are not to be construed in any way as limiting their scope.

### Example 1. Absence of Treatment-Related Sexual Dysfunction in the Phase 3, Randomized, Double-Blinded, Placebo-Controlled MOUNTAIN Study of Compound (1) in Patients With Major Depressive Disorder

### Introduction

Sexual dysfunction is a prevalent symptom of major depressive disorder (MDD), with symptoms typically emerging 1 to 3 weeks after treatment initiation. The estimated prevalence ranges from 4% to 73%, depending on the type of antidepressant administered (Jacobsen, et al. CNS Spectr. 2020; 25 (1):50-63; and Lorenz T, et al. Mayo Clin Proc. 2016; 91 (9):1280-1286). Dysregulated gamma-aminobutyric acid (GABA) signaling may contribute to the development of depression by disrupting adaptive signaling in key neuronal networks, such as those controlling mood, arousal, behavior, and cognition (Luscher B, et al. F1000Research. 2019; 8:751; Maguire J. Front Cell Neurosci. 2019; 13:83; Fogaça M V, et al. Front Cell Neurosci. 2019; 13:87; Fischer A S, et al. Biol Psychiatry Cogn Neurosci Neuroimaging. 2016; 1 (3):262-270; and Brown RE, et al. Front Neurol. 2015; 6:135). Neuroactive steroid binding to GABAA receptors results in potentiation of both synaptic and extrasynaptic GABAA receptor populations and leads to increased GABAA receptor surface expression. Extrasynaptic GABAA receptors containing delta subunits are insensitive to benzodiazepines (Althaus A L, et al. Neuropharmacology. 2020; 181:108333; and Martinez Botella G, et al. J Med Chem. 2017; 60 (18):7810-7819).

Compound (1) is an investigational, neuroactive steroid and GABA receptor positive allosteric modulator under evaluation as an oral, once-daily, 2-week therapy for MDD (Althaus A L, et al. Neuropharmacology. 2020; 181:108333; Martinez Botella G, et al. J Med Chem. 2017; 60 (18):7810-7819; Hoffmann E, et al. Clin Pharmacokinet. 2020; 59 (1):111-120; Sage Therapeutics, Inc. Data on file. MLIB-2982; and Clayton, A. H. Presented at SOBP 2020 Virtual Meeting).

Results from the multicenter, double-blind, randomized, placebo-controlled Phase 3 MOUNTAIN trial (NCT03672175) evaluating the efficacy, safety, and tolerability of 14-day treatment with 20 mg or 30 mg of Compound (1) compared with placebo (PBO) have been previously presented (Clayton, A. H. Presented at SOBP 2020 Virtual Meeting).

This analysis assessed the effect of 30 mg of Compound (1) treatment compared with PBO on sexual function, as measured by the Changes in Sexual Functioning Questionnaire-14 (CSFQ-14), in the MOUNTAIN trial.

### Methods

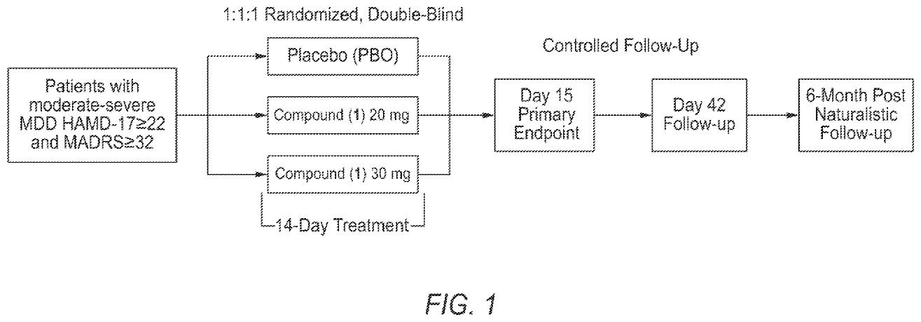

Adult outpatients (18-65 years; n=581) with MDD (HAMD-17 total score≥22 and MADRS total score≥32) were randomized 1:1:1 to Compound (1) 20 mg (data not shown), Compound (1) 30 mg, or PBO, once-nightly for 2 weeks, with follow-up through Day 42 (see FIG. 1 for Study Design). Standard of care antidepressants that had been taken at the same dose for ≥60 days prior to Day 1 of the trial were permitted. Exclusion criteria included attempted suicide associated with the current depressive episode; treatment-resistant depression, and medical history of bipolar disorder, schizophrenia, and/or schizoaffective disorder.

Exploratory, post-hoc analyses included evaluation of sexual functioning using the 14-item; self-report CSFQ-14 at baseline, Day 15, 28, and 42.

Male and female sexual functioning was evaluated as an exploratory endpoint with the validated, 14-item, patient-reported CSFQ-14 scores at baseline, Day 15, Day 28, and Day 42. CSFQ-14 also has 5 sub-scales that measure pleasure, desire/frequency, desire/interest, arousal/excitement, and orgasm/completion, which were also evaluated. Sexual dysfunction was defined as a CSFQ-14_{TS} of ≤41 for females and ≤47 for males. Mixed model for repeated measure and generalized estimating equation were used for analysis on patients with a baseline HAMD-17 total score≥24 (n=323).

### Results

There were 227 females and 96 males included in this analysis, e.g., with a baseline HAMD-17 total score≥24. At baseline, the mean baseline CSFQ-14_{TS} for Compound (1) 30 mg and PBO for female patients was 31.7 (n=121) and 33.7 (n=105), and for male patients was 42.1 (n=45) and 40.9 (n=51); respectively. All groups met the validated levels of sexual dysfunction at baseline.

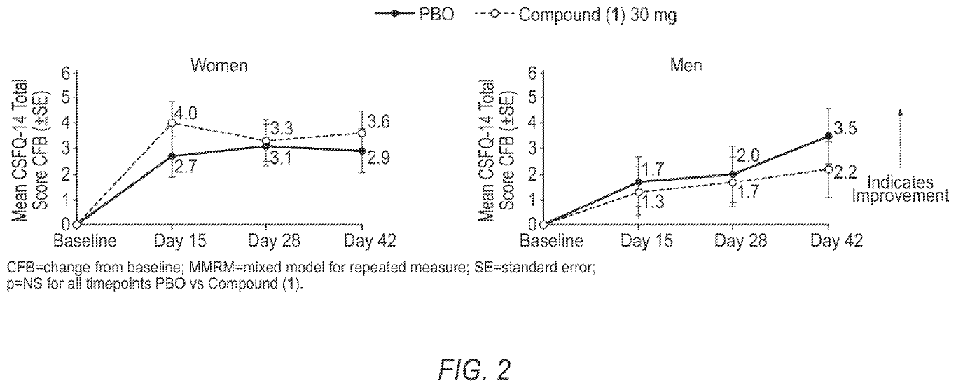

No impairment of sexual function compared to placebo was observed during the trial and there were no significant differences between Compound (1) 30 mg- and PBO-treated patients in least-square mean (standard error) change from baseline CSFQ-14_{TS} improvements in females at Day 15 (+4.0 [0.83] and +2.7 [0.74]), Day 28 (+3.3 [0.80] and +3.1 [0.82]) and Day 42 (+3.6 [0.87] and +2.9 [0.88]). No statistically significant differences were observed in males between treatment arms at Day 15 (+1.3 [1.28] and +1.7 [1.23]), Day 28 (+1.7 [1.31] and +2.0 [1.30]) and Day 42 (+2.2 [1.33] and +3.5 [1.54]), respectively. (See FIG. 2).

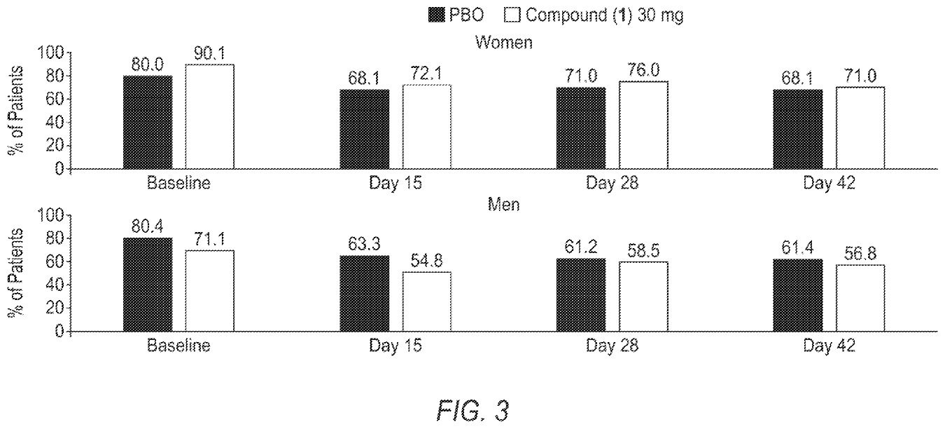

The percentage of all patients with sexual dysfunction as assessed by CSFQ-14_{TS} thresholds decreased from 82.4% (266/323) at baseline to 66.5% (181/272) at Day 42 for Compound (1) 30 mg- and PBO-treated patients respectively, with no significant difference in the odds ratio for sexual dysfunction between treatment arms. (See FIG. 3).

As shown in Table 1, below, there were no significant differences observed between Compound (1) 30 mg- and PBO-treated patients in the odds ratio for sexual dysfunction (e.g., CSFQ total score≤41 for women, ≤47 for men represents sexual dysfunction).

The model used was a generalized estimating equation (GEE) for binary response model, with factors for treatment (Compound (1) 30 mg or placebo), baseline CSFQ-14 total score, antidepressant use at baseline (Yes or No), assessment time point, and time point-by-treatment interaction with exchangeable covariance structure.

Odds ratio is the estimate of the odds of having CSFQ sexual dysfunction for subjects treated with Compound (1) relative to that for subjects treated with placebo.

TABLE 1
Odds Ratio for Sexual Dysfunction

| Time | Sex | Odds Ratio | 95% CI | P value |
|---|---|---|---|---|
| Day 15 | Women | 1.00 | 0.51, 1.94 | 0.9959 |
| | Men | 0.72 | 0.27, 1.91 | 0.5106 |
| Day 28 | Women | 1.01 | 0.50, 2.05 | 0.9706 |
| | Men | 1.07 | 0.41, 2.78 | 0.8895 |
| Day 42 | Women | 0.91 | 0.46, 1.81 | 0.7815 |
| | Men | 0.88 | 0.32, 2.41 | 0.8089 |

Additionally, there were no significant differences between Compound (1) 30 mg and PBO in the percentage of patients with sexual dysfunction as assessed in the individual CSFQ-14 subscales.

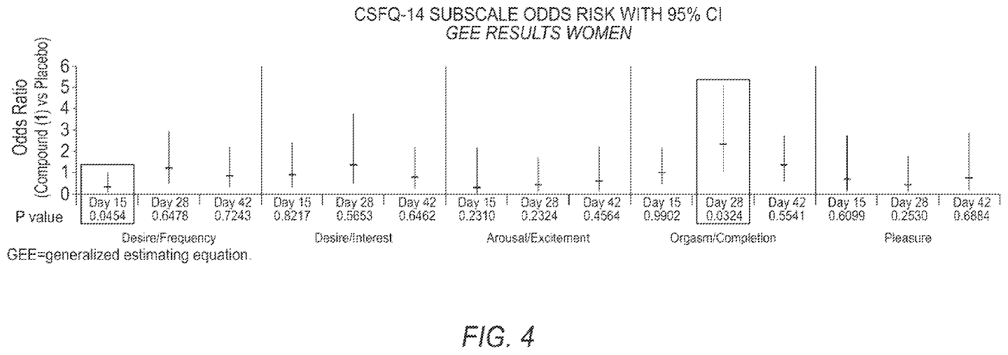

As shown in FIG. 4, no significant differences were observed between Compound (1) 30 mg and PBO in the percentage of female patients with sexual dysfunction across individual CSFQ-14 subscales, with the exception of desire/frequency at Day 15 and orgasm/completion at Day 28.

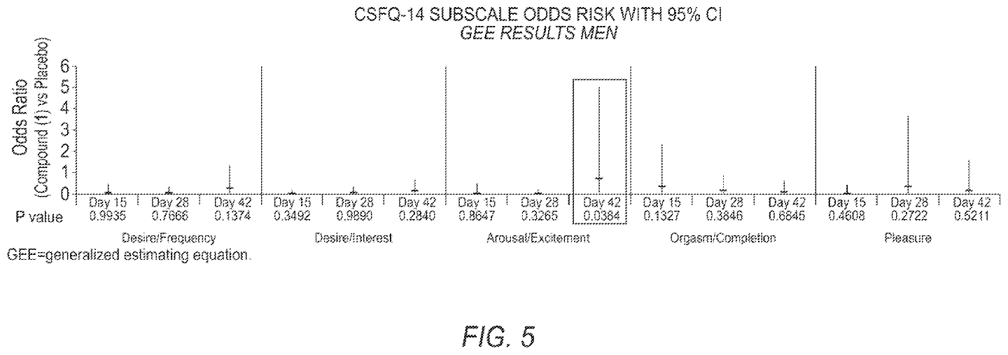

As shown in FIG. 5, no significant differences were observed between Compound (1) 30 mg and PBO in the percentage of male patients with sexual dysfunction across individual CSFQ-14 subscales, with the exception of arousal/excitement at Day 42.

### Conclusion

This post-hoc analysis of CSFQ-14_{TS} demonstrated no differences in sexual functioning between female or male patients who received Compound (1) 30 mg or PBO in the MOUNTAIN trial. Analysis of individual CSFQ-14 subscale scores also showed a similar trend with no consistent differences between patients who received Compound (1) 30 mg or PBO, regardless of sex.

These data suggest that treatment with Compound (1) 30 mg is not associated with treatment-emergent sexual dysfunction, a side effect associated with antidepressants. These overall findings support further development of Compound (1) for potential use as an as-needed, oral, once-daily 2-week treatment for MDD.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

### Other Embodiments

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating sexual dysfunction in a patient in need thereof, comprising administering to the patient a therapeutically effective amount of a compound of the formula selected from the group consisting of:

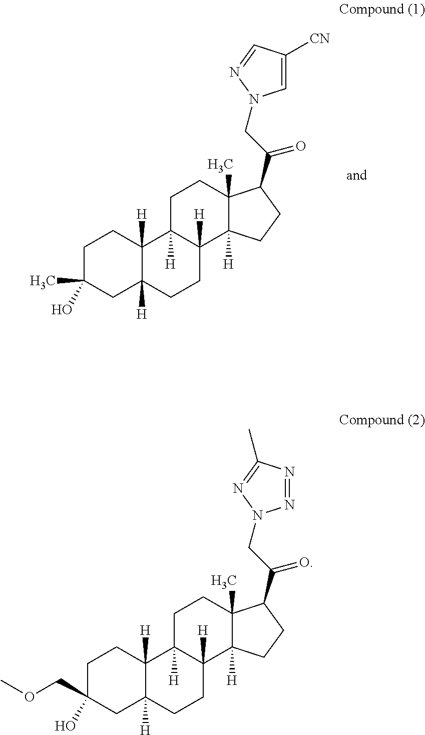

2. The method of claim 1, wherein the compound is a compound of the formula:

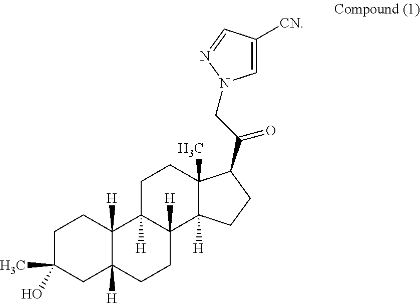

3. The method of claim 1, wherein the compound is a compound of the formula:

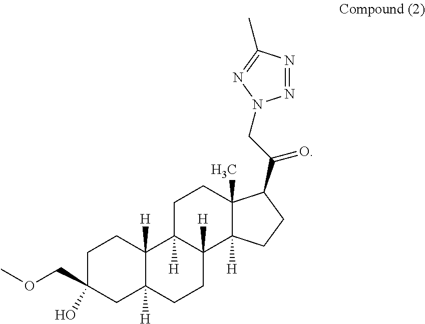

4. A method of treating sexual dysfunction in a patient in need thereof, comprising administering to the patient a therapeutically effective amount of a pharmaceutically acceptable salt of a compound of the formula selected from the group consisting of:

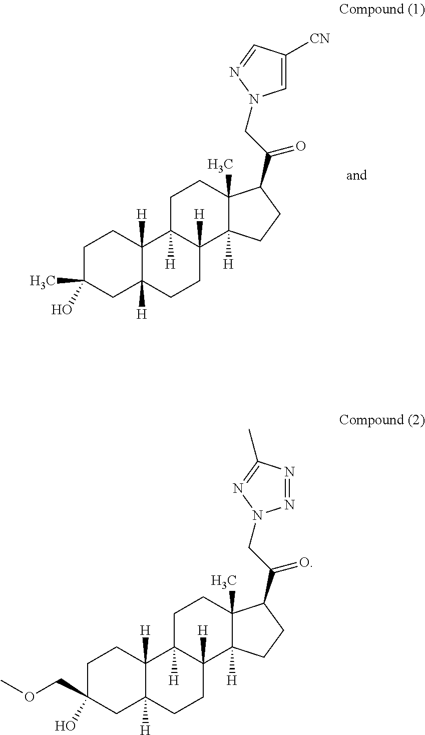

5. The method of claim 4, wherein the pharmaceutically acceptable salt is a pharmaceutically acceptable salt of a compound of the formula:

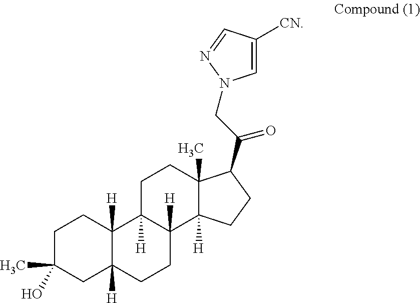

6. The method of claim 4, wherein the pharmaceutically acceptable salt is a pharmaceutically acceptable salt of a compound of the formula:

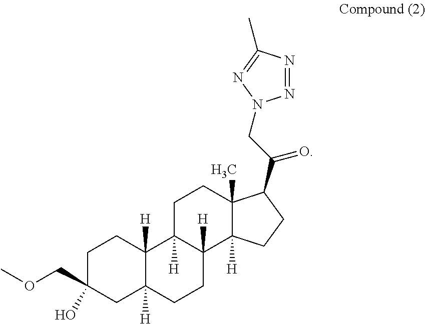

7. The method of any one of claims 1-6, wherein the patient has a CNS-related disorder.

8. The method of claim 7, wherein the CNS-related disorder is selected from the group consisting of a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, and status epilepticus.

9. The method of claim 8, wherein the CNS-related disorder is a mood disorder.

10. The method of claim 9, wherein the mood disorder is major depressive disorder.

11. The method of any one of claims 1-3, wherein the compound is administered at a dose of about 20 mg to about 60 mg once a day.

12. The method of claim 11, wherein the compound is administered at a dose of about 50 mg once a day for about 14 days.

13. The method of claim 11, wherein the compound is administered at a dose of about 60 mg once a day for about 14 days.

14. The method of any one of claims 4-6, wherein the pharmaceutically acceptable salt of the compound is administered at a dose equivalent to about 20 mg to about 60 mg of the free base compound once a day.

15. The method of claim 14, wherein the pharmaceutically acceptable salt of the compound is administered at a dose equivalent to about 50 mg of the free base compound once a day for about 14 days.

16. The method of claim 14, wherein the pharmaceutically acceptable salt of the compound is administered at a dose equivalent to about 60 mg of the free base compound once a day for about 14 days.

17. The method of any one of claims 1-16, wherein the sexual dysfunction comprises decrease in or loss of one or more of sexual pleasure, sexual desire, sexual frequency, sexual interest, sexual arousal, sexual excitement, and orgasm.

18. A method of treating sexual dysfunction in a patient having major depressive disorder, the method comprising administering a therapeutically effective amount of a compound of the formula selected from the group consisting of:

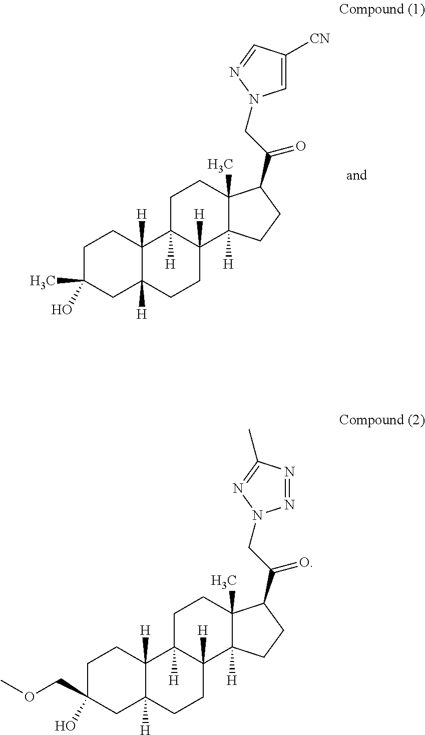

19. The method of claim 18, wherein the compound is a compound of the formula:

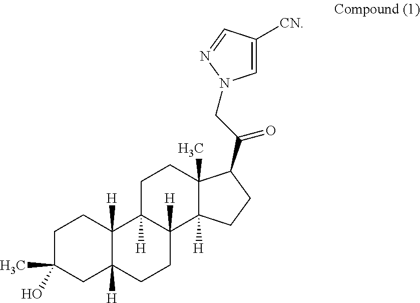

20. The method of claim 18, wherein the compound is a compound of the formula:

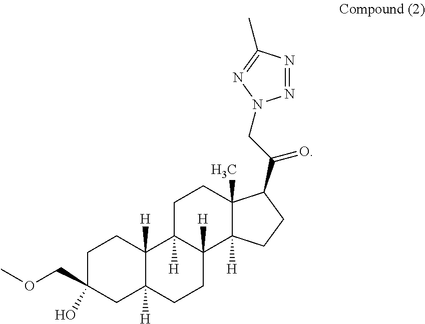

21. A method of treating sexual dysfunction in a patient having major depressive disorder, the method comprising administering a therapeutically effective amount of a pharmaceutically acceptable salt of a compound of the formula selected from the group consisting of:

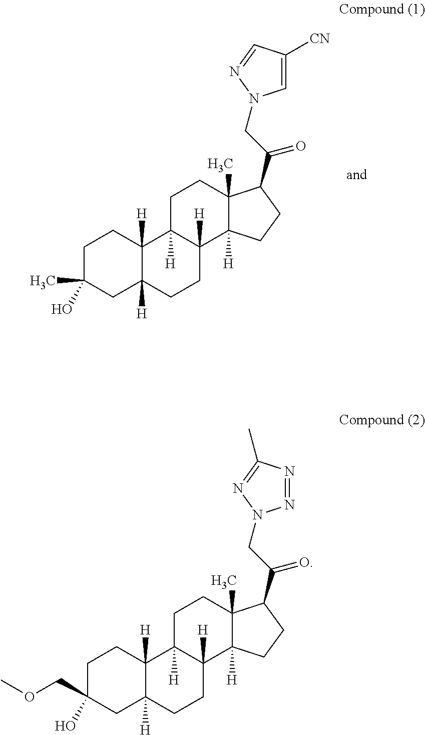

22. The method of claim 21, wherein the pharmaceutically acceptable salt is a pharmaceutically acceptable salt of a compound of the formula:

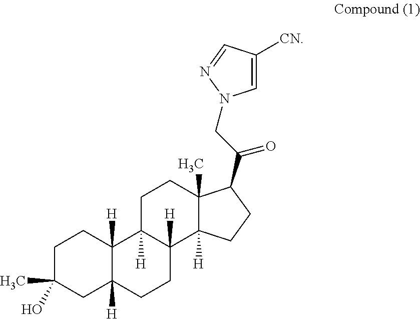

23. The method of claim 21, wherein the pharmaceutically acceptable salt is a pharmaceutically acceptable salt of a compound of the formula:

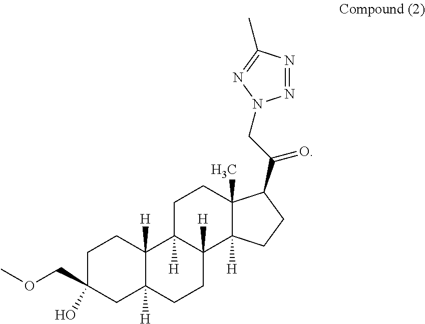

24. The method of any one of claims 18-20, wherein the compound is administered at a dose of about 20 mg to about 60 mg once a day.

25. The method of claim 24, wherein the compound is administered at a dose of about 50 mg once a day for about 14 days.

26. The method of claim 24, wherein the compound is administered at a dose of about 60 mg once a day for about 14 days.

27. The method of any one of claims 21-23, wherein the pharmaceutically acceptable salt of the compound is administered at a dose equivalent to about 20 mg to about 60 mg of the free base compound once a day.

28. The method of claim 27, wherein the pharmaceutically acceptable salt of the compound is administered at a dose equivalent to about 50 mg of the free base compound once a day for about 14 days.

29. The method of claim 27, wherein the pharmaceutically acceptable salt of the compound is administered at a dose equivalent to about 60 mg of the free base compound once a day for about 14 days.

30. The method of any one of claims 18-29, wherein the sexual dysfunction comprises decrease in or loss of one or more of sexual pleasure, sexual desire, sexual frequency, sexual interest, sexual arousal, sexual excitement, and orgasm.

31. A method of maintaining sexual function in a patient who has a mood disorder, comprising administering to the patient a therapeutically effective amount of a compound of the formula selected from the group consisting of:

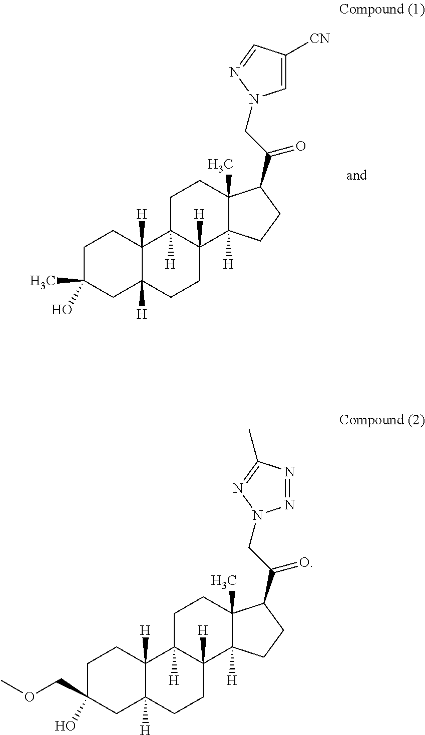

32. The method of claim 31, wherein the compound is a compound of the formula:

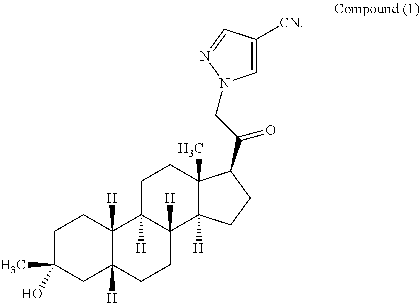

33. The method of claim 31, wherein the compound is a compound of the formula:

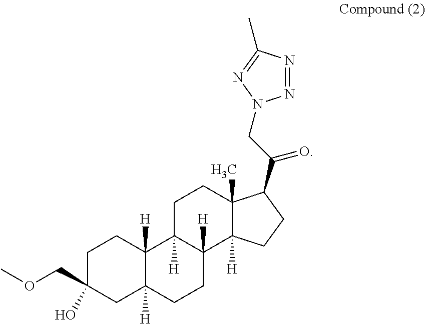

34. A method of maintaining sexual function in a patient who has a mood disorder, comprising administering to the patient a therapeutically effective amount of a pharmaceutically acceptable salt of a compound of the formula selected from the group consisting of:

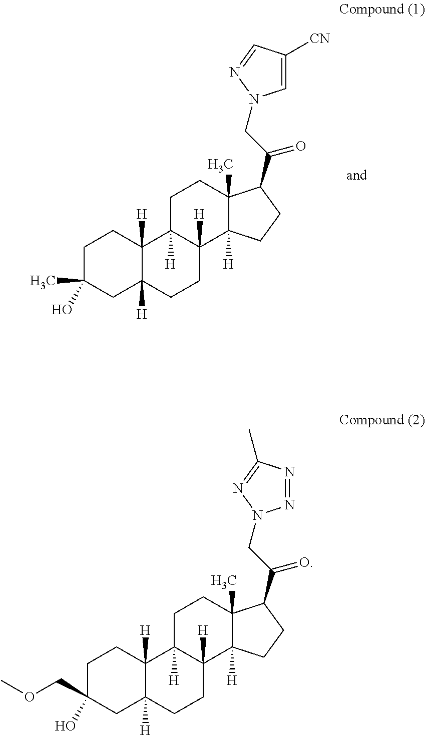

35. The method of claim 34, wherein the pharmaceutically acceptable salt is a pharmaceutically acceptable salt of a compound of the formula:

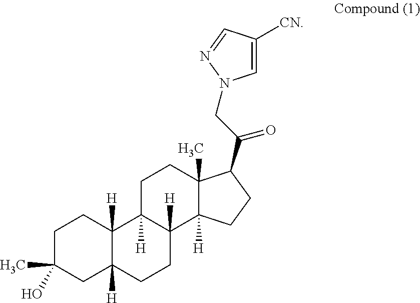

36. The method of claim 34, wherein the pharmaceutically acceptable salt is a pharmaceutically acceptable salt of a compound of the formula:

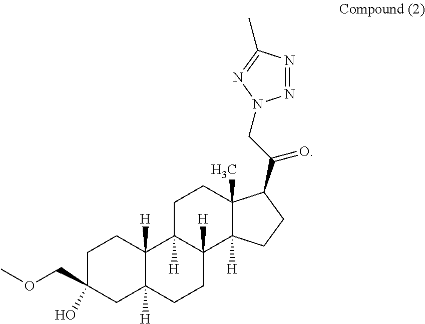

37. The method of any one of claims 31-36, wherein the mood disorder is major depressive disorder.

38. The method of any one of claims 31-33, wherein the compound is administered at a dose of about 20 mg to about 60 mg once a day.

39. The method of claim 38, wherein the compound is administered at a dose of about 50 mg once a day for about 14 days.

40. The method of claim 38, wherein the compound is administered at a dose of about 60 mg once a day for about 14 days.

41. The method of any one of claims 34-36, wherein the pharmaceutically acceptable salt of the compound is administered at a dose equivalent to about 20 mg to about 60 mg of the free base compound once a day.

42. The method of claim 41, wherein the pharmaceutically acceptable salt of the compound of the formula is administered at a dose equivalent to about 50 mg of the free base compound once a day for about 14 days.

43. The method of claim 41, wherein the pharmaceutically acceptable salt of the compound of the formula is administered at a dose equivalent to about 60 mg of the free base compound once a day for about 14 days.

44. The method of any one of claims 31-43, wherein the sexual function comprises one or more of sexual pleasure, sexual desire, sexual frequency, sexual interest, sexual arousal, sexual excitement, and orgasm.

45. A method of treating treatment-induced sexual dysfunction in a patient who is currently receiving or has received a course of chemical psychotherapy, comprising administering to the patient a therapeutically effective amount of a compound of the formula selected from the group consisting of:

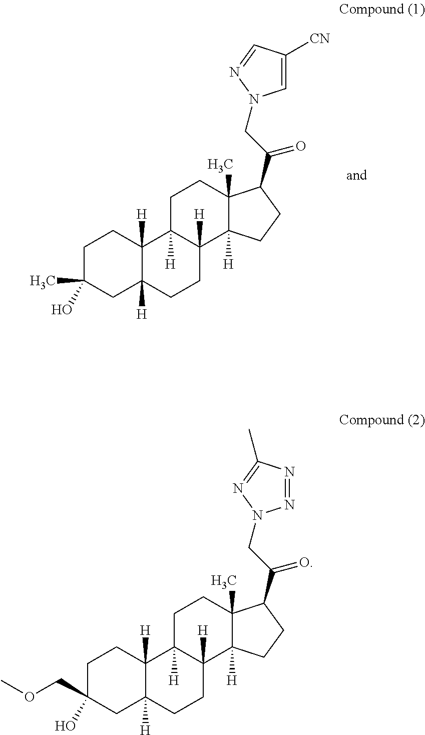

46. The method of claim 45, wherein the compound is a compound of the formula:

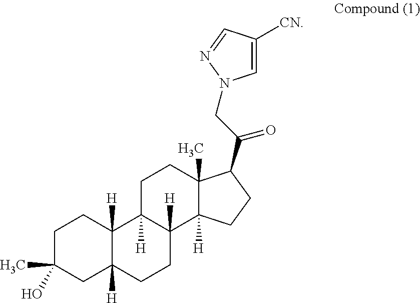

47. The method of claim 45, wherein the compound is a compound of the formula:

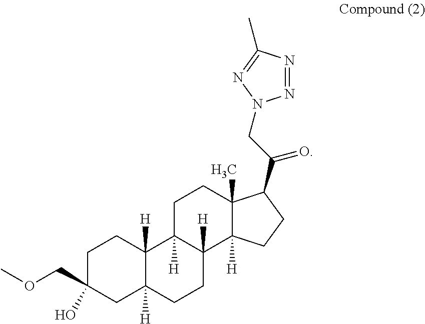

48. A method of treating treatment-induced sexual dysfunction in a patient who is currently receiving or has received a course of chemical psychotherapy, comprising administering to the patient a therapeutically effective amount of a pharmaceutically acceptable salt of a compound of the formula selected from the group consisting of:

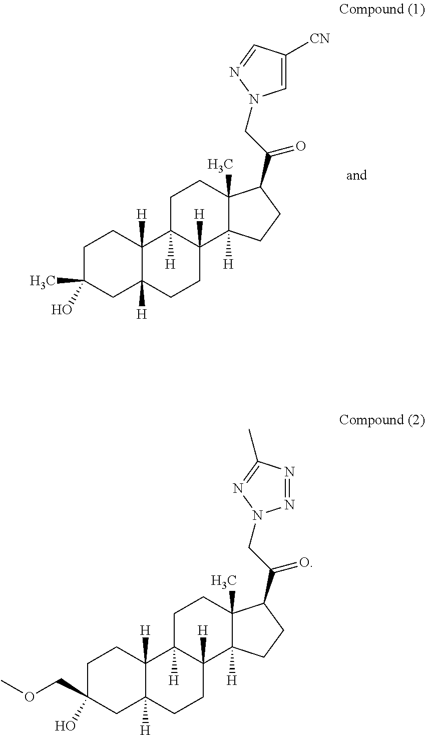

49. The method of claim 48, wherein the pharmaceutically acceptable salt is a pharmaceutically acceptable salt of a compound of the formula:

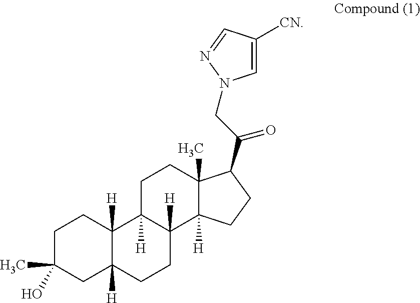

50. The method of claim 48, wherein the pharmaceutically acceptable salt is a pharmaceutically acceptable salt of a compound of the formula:

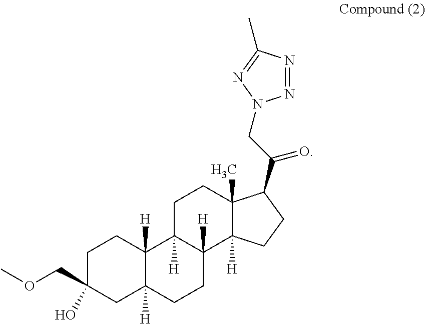

51. The method of any one of claims 45-50, wherein the chemical psychotherapy is an antidepressant.

52. The method of claim 51, wherein the antidepressant is selected from a selective serotonin reuptake inhibitor, a selective norepinephrine reuptake inhibitor, a tricyclic, a monoamine oxidase inhibitor, or an atypical antidepressant.

53. The method of any one of claims 45-52, wherein the patient has a CNS-related disorder.

54. The method of claim 53, wherein the CNS-related disorder is selected from the group consisting of a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, and status epilepticus.

55. The method of claim 54, wherein the CNS-related disorder is a mood disorder.

56. The method of claim 55, wherein the mood disorder is major depressive disorder.

57. The method of any one of claims 45-47, wherein the compound is administered at a dose of about 20 mg to about 60 mg once a day.

58. The method of claim 57, wherein the compound is administered at a dose of about 50 mg once a day for about 14 days.

59. The method of claim 57, wherein the compound is administered at a dose of about 60 mg once a day for about 14 days.

60. The method of any one of claims 48-50, wherein the pharmaceutically acceptable salt of the compound is administered at a dose equivalent to about 20 mg to about 60 mg of the free base compound once a day.

61. The method of claim 60, wherein the pharmaceutically acceptable salt of the compound is administered at a dose equivalent to about 50 mg of the free base compound once a day for about 14 days.

62. The method of claim 60, wherein the pharmaceutically acceptable salt of the compound is administered at a dose equivalent to about 60 mg of the free base compound once a day for about 14 days.

63. The method of any one of claims 41-62, wherein the sexual dysfunction comprises decrease in or loss of one or more of sexual pleasure, sexual desire, sexual frequency, sexual interest, sexual arousal, sexual excitement, and orgasm.
